(19) United States
(12) Patent Application Publication
(10) Pub. No.: US 2024/0337655 A1
(43) Pub. Date: Oct. 10, 2024

(54) SILK FIBROIN TO STABILIZE "DE NOVO" SENSING PROTEINS

(71) Applicants: Trustees of Tufts College, MEDFORD, MA (US); University of Washington, Seattle, WA (US)

(72) Inventors: Fiorenzo G. Omenetto, Lexington, MA (US); Luciana d'Amone, Washington, DC (US); Giuseppina Matzeu, Medford, MA (US); Alfredo Quijano Rubio, Seattle, WA (US); David Baker, Seattle, WA (US)

(21) Appl. No.: 18/597,315

(22) Filed: Mar. 6, 2024

Related U.S. Application Data
(60) Provisional application No. 63/488,687, filed on Mar. 6, 2023.

(51) Int. Cl. G01N 33/566; G01N 33/569; G01N 33/573; G01N 33/74

(52) U.S. Cl. CPC G01N 33/566; G01N 33/56983; G01N 33/573; G01N 33/74; G01N 2333/43526

(57) ABSTRACT

A solid form sensing article can have one or more silk fibroin matrix sensing units, each having a solid form sensing property. Each of the one or more silk fibroin matrix sensing units can include a silk fibroin matrix. A plurality of Cage proteins and a plurality of Key proteins can be entrained within the silk fibroin matrix. The Cage proteins can bind to an analyte of interest.

### CROSS-REFERENCE TO RELATED APPLICATIONS

This application relates to, incorporates by reference for all purposes, and claims priority to U.S. Application Ser. No. 63/488,687, filed Mar. 6, 2023.

### STATEMENT REGARDING FEDERALLY SPONSORED RESEARCH

This invention was made with government support under grant N00014-19-1-2399 awarded by the Office of Naval Research. The government has certain rights in the invention.

### FIELD OF THE INVENTION

The disclosed technology is generally directed to biosensing articles. More particularly the technology is directed to biosensing articles that provide a luminescent signal.

### BACKGROUND OF THE INVENTION

Sensing devices that can identify biomarkers at low concentration levels are needed in public spaces, personal care items, and especially in clinical environs. Biosensors often rely on complex transduction mechanisms, however robust detection mechanisms are needed. Refined methods based on biochemical transduction agents such as RNA or antibodies have proven effective, however the processing and shelf-stability of these formats are often encumbered by challenges associated with sample treatment and logistics of test result readout. New sensors should have minimal sample treatment requirements, easy-to-read results, as well as shelf-stability, portability, and modular stability of the biochemical components. Sensor stability is especially needed for extended periods in the various conditions encountered during manufacturing, storage, and transportation, such as exposure to high/low temperatures, temperature fluctuations, and humidity.

### BRIEF SUMMARY OF THE INVENTION

De novo designed protein switches are powerful tools for specifically and sensitively detecting diverse targets with simple chemiluminescent readouts. Finding an appropriate material host to entrain these protein switches without altering their thermodynamics enables a variety of sensing formats to monitor exposure to pathogens, toxins, or for disease diagnosis. Disclosed herein is a de novo protein-biopolymer hybrid that maintains the detection capabilities induced by the structural change of the incorporated proteins in response to analytes of interest in multiple, shelf-stable material formats. A set of functional demonstrator devices including personal protective equipment such as masks and laboratory gloves, free-standing films, air quality monitors, and wearable devices are presented to illustrate the versatility of the approach. Such formats are designed to be responsive to human epidermal growth factor receptor (HER2), Hepatitis B (HBV), Botulinum neurotoxin B (BoNT/B), and severe acute respiratory syndrome coronavirus 2 (SARS-CoV-2), This rationally designed combination of form and function offers wide opportunities for ubiquitous sensing in multiple environments by enabling a large class of bio-responsive interfaces of broad utility.

Accordingly, aspects of the present invention provide for a solid form sensing article comprising one or more silk fibroin matrix sensing units, wherein each of the one or more silk fibroin matrix sensing units comprises a silk fibroin matrix having entrained therein a plurality of Cage proteins and a plurality of Key proteins which form part of a Cage-Key protein system. Each of the one or more silk fibroin matrix sensing units has a solid form sensing property as measured when exposing the respective silk fibroin matrix sensing unit to an analyte of interest. The plurality of Cage proteins and the plurality of Key proteins have a spatial distribution within the silk fibroin matrix that is selected from the group consisting of homogeneous distribution, random distribution, pseudo-random distribution, and combinations thereof.

Another aspect provided herein is a method of using the solid form sensing article, where the method comprises monitoring a Cage-Key signal associated with the presence of the analyte of interest.

Another aspect provided herein is a method of making a solid form sensing article comprising: 1) drop casting liquid silk fibroin-key/cage matrices converted into sensitive films after water evaporation; 2) lyophilizing silk fibroin-key/cage matrices to make sponges; 3) electrospinning silk fibroin-key/cage matrices to make non-woven mats; and/or 4) inkjet printing and dispensing pico and nanodrops of silk fibroin-key/cage matrices via digitally controlled interfaces on different substrates (including stretchable and flexible fabrics, rigid polymeric surfaces and common objects) to generate a variety of printed patterns.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 Fabrication of silk fibroin biosensing formats for universal biomarker detection. Protein switches (lucKey-lucCage, or Key-Cage protein systems) that enable the detection of SARS-COV-2 spike protein (RBD), HER2 receptor (HER2), Hepatitis B (anti-HBV), and Botulinum Neurotoxin B (BoNT/B), are combined with regenerated silk fibroin to fabricate a library of bio-active platforms, comprising free standing sponges and films, electrospun non-woven mats, bio-active personal protective equipment (mask and glove), jar lids, and breast pads capable of responding to a wide range of targets.

FIG. 2 Calibration and characterization of silk sponges and films for biomarkers detection. a, Schematic representation of the characterization and stability studies procedures of doped silk fibroin films and sponges. b, d, f, h Luminescence response of doped silk fibroin sponges prepared with 2% silk fibroin and 5 nM lucKey-lucCageHER2 (b), 25 nM lucKey-lucKeyBoNT/B (d), 5 nM lucKey-lucCageRBD (f), or 6.25 nM lucKey-lucCageHBV (h) after dissolution and exposure at various concentrations of analytes. c, e, g, i Bioluminescent response (reported as gray value) of doped RSF films prepared with 2% silk fibroin 5 nM lucKey-lucCageHER2 (c), 2% silk fibroin 25 nM lucKey-lucKeyBoNT/B (e), 4% silk fibroin 5 nM lucKey-lucCageRBD (g), and 2% silk fibroin 6.25 nM lucKey-lucCageHBV (i) as a function of target concentration. Insets represent the corresponding bioluminescence images in false color. (1) Analyte specificity of lucKey-lucCageRBD film. Schematic representation of testing procedure (top), photograph of silk fibroin film (inset), and bioluminescence images (bottom) of 2% silk fibroin 5 nM lucKey-lucCageRBD recorded in presence of RBD target and potential interfering analytes (HER2, anti-HBV, and BoNT/B).

FIG. 3 Luminescence characterization of HER2-responsive silk sponges. Luminescence kinetics (A, C, E) and corresponding normalized luminescence (B, D, F) as a function of HER2 target concentration of 4% silk fibroin sponges embedding 1 nM (A, B) or 5 nM (C, D) of lucKey-lucCageHER2, and 2% silk fibroin embedding 1 nM (E, F) of lucKey-lucCageHER2 after exposure at various concentrations of HER2 ectodomain protein (0.04-125 nM). Detecting performances were extrapolated from the luminescence kinetics at 102 minutes (B, D) or 51 minutes (F).

FIG. 4 Luminescence characterization of RBD-responsive silk sponges. Luminescence kinetics (A, C) and corresponding normalized luminescence (B, D) as a function of SARS-CoV-2 RBD target concentration of 4% silk fibroin sponges embedding 5 nM (A, B) of lucKey-lucCageRBD, and 2% silk fibroin embedding 1 nM (C, D) of lucKey-lucCageRBD after exposure at various concentration of RBD (0.4-150 pM). Detecting performances were extrapolated from the luminescence kinetics at 51 minutes (B, D).

FIG. 5 Luminescence characterization of BoNT/B-responsive silk sponges. Luminescence kinetics (A) and corresponding normalized luminescence as a function of BoNT/B target concentration (B) of 4% silk fibroin sponges embedding 25 nM of lucKey-lucCageBot after exposure at various concentrations of BoNT/B (0.064-200 nM). Detecting performances were extrapolated from the luminescence kinetics at 51 minutes (B).

FIG. 6 Luminescence characterization of anti-HBV antibody-responsive silk sponges. Normalized luminescence as a function of antiHBV antibody concentration of 2% silk fibroin sponges embedding 6.25 nM of lucKey-lucCageHBVa after exposure at various concentrations of anti-hepatitis B virus antibody (0.064-40 nM). Detecting performances were extrapolated from the luminescence kinetics at 51 minutes.

FIG. 7. Luminescence characterization of HER2-responsive silk films. Luminescence kinetics (A, C, E, G) and corresponding normalized luminescence as a function of HER2 concentration (B, D, F, H) of 4% silk fibroin films embedding 1 nM (A, B) or 5 nM (C, D) of lucKey-lucCageHER2, and 2% silk fibroin embedding 1 nM (E, F) or 5 nM (G, H) of lucKey-lucCageHER2 after exposure at various concentrations of HER2 ectodomain protein (0.04-125 nM). Detecting performances were extrapolated from the luminescence kinetics at 102 minutes (B, D, F) or 51 minutes (H).

FIG. 8. Luminescence characterization of RBD-responsive silk films. (A) Normalized luminescence as a function of RBD concentration of lucKey-lucCageRBD in 2% silk solution after exposure at various concentrations of RBD (2-1250 pM) (in liquid control). Luminescence kinetics (B, D) and corresponding normalized luminescence as a function of RBD concentration (C, E) of 2% silk fibroin films at 1 nM (B, C) or 5 nM (D, E) and 4% silk fibroin films at 5 nM (F, G) of lucKey-lucCageRBD after exposure at various concentrations of RBD (0.4-150 pM). Detecting performances were extrapolated from the luminescence kinetics at 120 (A), 20 (C), or 51 minutes (E, G).

FIG. 9 Luminescence characterization of BoNT/B-responsive silk films. (A) Normalized luminescence as a function of BoNT/B concentration of lucKey-lucCageBot in 4% silk solution after exposure at various concentrations of BoNT/B (0.032-200 nM) (in liquid control). Luminescence kinetics (B, D) and corresponding normalized luminescence (C, E) as a function of BoNT/B concentration of 4% silk fibroin films (B, C) and 2% silk fibroin (D, E) embedding 25 nM of lucKey-lucCageBot after exposure at various concentrations of BoNT/B (0.064-200 nM). Detecting performances were extrapolated from the luminescence kinetics at 120 (A) and 24 minutes (C, E).

FIG. 10 Luminescence characterization of anti-HBV antibody-responsive silk films. (A) Normalized luminescence as a function of anti-hepatitis B virus antibody concentration of lucKey-lucCageHBV in 2% silk solution after exposure at various concentrations of anti-hepatitis B virus antibody (1.6-200 nM) (in liquid control). Luminescence kinetics (B, D) and corresponding normalized luminescence (C, E) as a function of target concentration of 4% (B, C) and 2% (D, E) silk fibroin films embedding 6.25 nM of lucKey-lucCageHBV after exposure at various concentrations of anti-hepatitis B virus antibody (0.064-200 nM for A-B, 0.064-40 nM for D-E). Detecting performances were extrapolated from the luminescence kinetics at 120 (A) and 24 minutes (C, E).

FIG. 11 Functionalization of personal protective equipment and electrospun mats. a, Schematic representation of processing steps for RBD detection on bioactive surfaces. b, Functionalized breast pads (left) can be used for HER2 detection in nipple fluids (right). c, Bioluminescence images of the functionalized breast pad in false color after exposure at various HER2 target concentrations (50 nM, 250 nM, 500 nM, and 12.5 nM clockwise from the top left). d, Functionalized lids can detect jars contaminated with BoNT/B. e, Bioluminescence images of the functionalized jar lid in false color after exposure at two BoNT/B concentrations (125, 500, and 500 nM clockwise from top right). f, Functionalized surgical mask for the detection of the RBD spike protein (top) and bioluminescence images along the printed pattern in false color (bottom) after exposure to 5 nM (left side of the mask) and 30 nM (right side of the mask) RBD concentration. g, Silk-functionalized ink printed on a nitrile glove (top) with the zoom of the pattern (inset), and bioluminescence images along the printed pattern in false color after exposure at various RBD concentrations (50, 12.5, 1.25, 0.25 nM from left to right). h, Prototype of a small drone carrying a silk sponge for the detection of RBD in contaminated air. i, Luminescence response of a 2% RSF 25 nM lucKey-lucCageRBD after exposure at vapor with 500 pM RBD target against control (water).

FIG. 12 Bioimaging characterization of RBD-responsive silk films and non-woven mat. (A) Chemiluminescent signals plotted as the average pixel intensities of the imaged sensing areas and respective chemiluminescent responses in false color (insert) of 25 nM lucKey-lucCageRBD films after exposure at furimazine and various target concentrations in water. (B) Photograph of an electrospun mat (top left), corresponding SEM micrograph (top right), and bioluminescence response (bottom) in false color in presence of increasing concentrations of RBD (2-1250 pM from left to right).

FIG. 13 Bioimaging of functional biomaterials (A) Bioluminescence images of the surgical mask along the printed pattern in false color after exposure to 5 nM (left side of the mask) and 30 nM (right side of the mask) RBD concentration (top) and luminescence response reported as gray value (bottom). (B) Bioluminescence images of a nitrile glove along the printed pattern in false color (top) and luminescence response at various RBD concentrations (50, 12.5, 1.25, 0.25 nM from left to right) reported as gray value (bottom). (C) Bioluminescence images of the functionalized breast pad in false color (right) and luminescence response at various Her2 target concentrations (50 nM, 250 nM, 500 nM and 12.5 nM clockwise from the top left) reported as gray value (left). Higher target concentration and merging of multiple drops during sample analysis led to increasing luminescence intensity. (D) Bioluminescence images of the functionalized jar lid in false color (top) and luminescence response at different BoNT/B concentrations (500, 300, 125 nM from left to right) reported as gray value (bottom).

FIG. 14 Accelerated aging tests of RBD-responsive silk sponges and films. Luminescence kinetics (A, B) and corresponding normalized luminescence (C, D) of 4% silk fibroin (A, C) and 2% silk fibroin sponges (B, D) embedding 5 nM of lucKey-lucCageRBD after exposure to 50 pM (A) or 150 pM (B) of RBD at different incubation times. Tables report the corresponding LODs for sponges (E) and films (F).

FIG. 15 Stability studies of HER2-responsive silk sponges. Luminescence kinetics (A) and corresponding normalized luminescence (B) of 2% silk fibroin sponges embedding 5 nM of lucKey-lucCageHER2 stored at room temperature for one year after exposure at furimazine and various concentrations of HER2 (0.2-125 nM).

FIG. 16 Effect of silk fibroin concentration on HER2 detection. Luminescence intensities at various target and regenerated silk fibroin concentrations at 0.5 nM (A), 1 nM (B), and 5 nM (C) of lucKey-lucCageHER2.

FIG. 17 Responsiveness of RBD-sensing complex to the Omicron variant target. Luminescence kinetics (A) and corresponding normalized luminescence (B) as a function of SARS-CoV-2 (Omicron variant) concentration of a 2% silk solution with 10 nM lucKey-lucCageRBD. Detecting performances were extrapolated from the luminescence kinetics at 120 minutes (B).

### DETAILED DESCRIPTION OF THE INVENTION

Before example embodiments of an apparatus in accordance with the disclosure are described in further detail, it is to be understood that the disclosure is not limited to the particular aspects described. It is also to be understood that the terminology used herein is for the purpose of describing particular aspects only and is not intended to be limiting. The scope of an invention described in this disclosure will be limited only by the claims. As used herein, the singular forms “a”, “an”, and “the” include plural aspects unless the context clearly dictates otherwise. It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Aspects referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise. Numeric ranges disclosed herein are inclusive of their endpoints. For example, a numeric range of between 1 and 10 includes the values 1 and 10. When a series of numeric ranges are disclosed for a given value, this disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

De novo designed protein-based biosensors have been recently demonstrated to be a versatile, reconfigurable biochemical transduction mechanism for sensing applications. In these systems, the sensing function is provided by the synergy of two designed protein components, a lucCage (hereinafter “a Cage protein”) and a lucKey (hereinafter “a Key protein”). The Cage protein includes an analyte binding domain. The analyte-binding domain can be de novo designed to specifically bind to an analyte of interest, thus allowing the Cage-Key system to be tuned to the analyte of interest. Proteins can be designed de novo to bind to a target protein with high specificity and affinity by using computational algorithms that have access to an exponentially larger protein sequence space than what is available in Nature. In contrast with naturally occurring proteins, which can bind a limited repertoire of molecules, de novo protein design enables the production of a wide variety of biosensors for any analytes with known structure.

When an analyte of interest interacts with the analyte binding domain, the Cage protein undergoes a conformational change from a closed state to an open state. In the open state, the Key protein forms a complex with the Cage protein. The Key protein includes a key peptide sequence that binds to the open state of the Cage protein and includes a complementary split luciferase fragment. The association of the Key protein and the Cage protein results in reconstitution of luciferase activity. The ensuing luciferase bioluminescence provides a rapid, specific, and sensitive readout of the analyte-driven Cage-Key protein system as a protein switch.

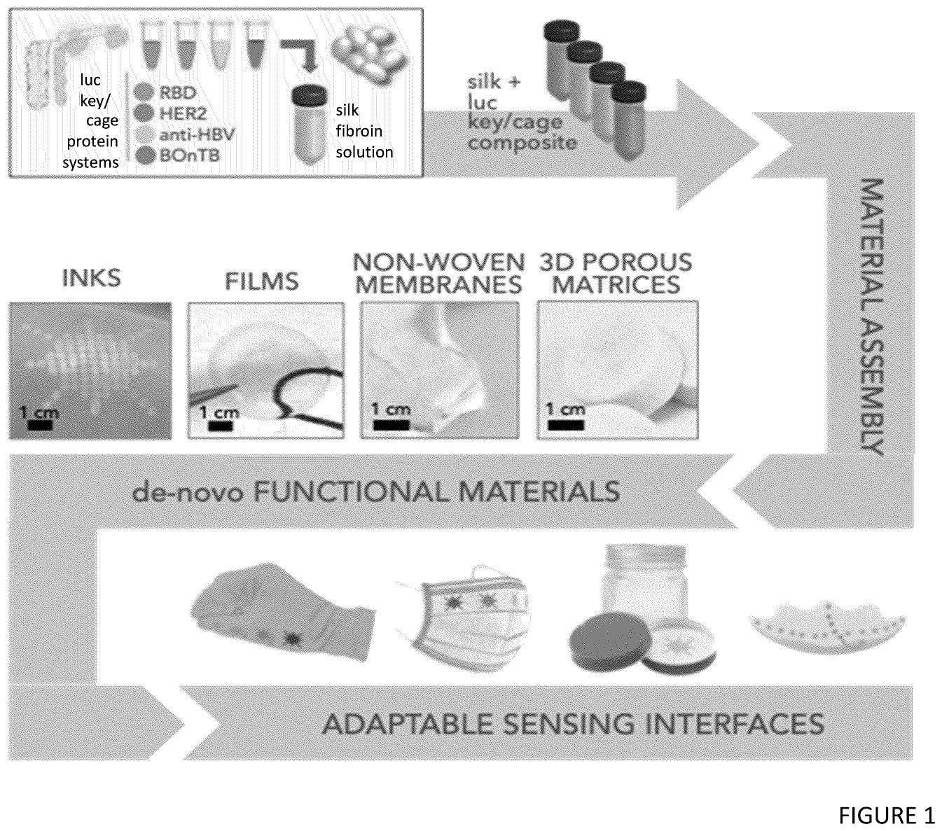

Disclosed herein are bio-responsive sensing materials of broad utility, described in more detail below and shown in FIG. 1. These bio-responsive materials incorporate several Key-Cage protein systems into a matrix. The matrix can be used to assemble de novo functional materials and adaptable sensing interfaces. The integration of dynamic, responsive protein switches through various fabrication strategies provides smart substrates designed to respond to (i) the receptor-binding domain (RBD) in Severe Acute Respiratory Syndrome Coronavirus 2 (SARS-CoV-2) spike protein, (ii) the Human Epidermal Growth Factor Receptor 2 (HER2), (iii) the Botulinum Neurotoxin B (BoNT/B), and (iv) the Anti-Hepatitis B Virus antibody (anti-HBV).

According to an aspect as disclosed herein, the analyte of interest can be the receptor-binding domain (RBD) in Severe Acute Respiratory Syndrome Coronavirus 2 (SARS-CoV-2) spike protein. In an aspect, the analyte of interest can be the Human Epidermal Growth Factor Receptor 2 (HER2). In another aspect, the analyte of interest can be the Botulinum Neurotoxin B (BoNT/B). In a further aspect, the analyte of interest can be the Anti-Hepatitis B Virus antibody (anti-HBV). In further aspects, the analyte of interest can be cortisol or amylase.

When the Cage-Key protein system is expressed by or contained within a cell, the Cage-Key protein system has an intracellular sensing property. The intracellular sensing property can be measured when exposing the cell expressing or containing the Cage-Key protein system to the analyte of interest. The intracellular sensing property can be a luminescent signal. The intensity of the luminescent signal can be related to the concentration of the Cage-Key protein system, the concentration of the analyte of interest, or both. The intracellular sensing property can indicate the sensitivity of the Cage-Key protein system to the analyte of interest when the Cage-Key protein system is contained within a cell.

When the Cage-Key protein system is dissolved in a solution, the Cage-Key protein system has a solution-phase sensing property. The solution-phase sensing property can be measured when exposing the solution to the analyte of interest. The solution-phase sensing property can be a luminescent signal. In the absence of the analyte of interest a small basal luminescent signal is obtained. When used with furimazine, a substrate, Cage-Key protein system produces a high-intensity luminescence when the analyte is present. This signal increases proportionally to the concentration of analyte of interest bound Cage-Key protein system. The solution-phase sensing property can indicate the sensitivity of the Cage-Key protein system to the analyte of interest when the Cage-Key protein system is dissolved. Alternatively, and additionally, furimazine analogues can be used to produce the luminescent signal, for example, hydrofurimazine, fluorofurimazine, or coelenterazine (CTZ). When the Cage and Key components are present at 0.1 or 1 nanomolar, the detectable range of analyte spans from 10 picomolar to 10 nanomolar or higher.

### Matrix

Despite the higher intrinsic stability of de novo protein switches such as the Key-Cage protein system in comparison with natural proteins, they are commonly stored in refrigerated conditions after snap-freezing or in lyophilized formats. Lyophilization within a stabilizing matrix (such as sugars or polyols) is a common method used to increase the thermal stability of labile molecules by reducing their molecular mobility and degradation processes.

According to an aspect herein, the plurality of Key proteins and the plurality of Cage proteins can be entrained within a matrix. Generally, a plurality of Key proteins, a plurality of Cage proteins, or a plurality of Key-Cage protein systems can be combined with a biopolymer to make a protein-biopolymer hybrid. In a particular example, the matrix can be a silk fibroin biopolymer derived from the native Bombyx mori silkworm fibers. Silk fibroin is a promising matrix for de novo protein switches due to its aqueous solubility, non-toxicity, green chemistry, and polymorphic features.

As shown in FIG. 1, a plurality of Key proteins and a plurality of Cage proteins that are de novo designed to specifically bind to a target can be dissolved with silk fibroin in a matrix solution. The concentration of the Cage proteins and the Key proteins in the silk fibroin matrix can be between 1-50 nM. For example, matrix solutions of 1 nM Key protein can be used to prepare HER2 and RBD sensitive sponges and films. In another example, matrix solutions of 6.25 nM Key protein can be used to prepare Anti-HBV sensitive sponges and films. In yet another example, matrix solutions of 25 nM Key protein can be used to prepare BoNT/B sensitive sponges and films. In a further example, matrix solutions of 25 nM Key protein can be used to prepare RBD sensitive electrospun mats. In a still further example, matrix solutions of 25 nM Key protein can be used to prepare printable inks for sensing. The concentration of the Cage protein in the matrix solutions can be the same as the concentration of the Key protein. Alternatively, the concentration of the Cage protein in the matrix solutions can be different from the concentration of the Key protein.

The Cage proteins and the Key proteins can be present in the silk fibroin matrix in a ratio of: 0.13-1.3% w/w for the Cage protein, 0.082-0.82% w/w for the Key protein, 22.5-80% w/w for the silk fibroin considering the total weight of the initial mix where the remainder of the solution is water or water-based, such as a buffer.

The solutions containing the silk fibroin and the plurality of Key proteins and the plurality of Cage proteins can be used to generate a silk fibroin matrix. The distribution of the plurality of Cage proteins and the plurality of Key proteins in the silk fibroin matrix can be a homogeneous distribution, a random distribution, a pseudo-random distribution, or combinations thereof.

In another example, the matrix can include a paper substrate, where the plurality of Key proteins and the plurality of Cage proteins are entrained within pulp or fibers of the paper substrate. In yet another example, the matrix can include sugars or polyols.

### Material Format

The silk fibroin matrix can be associated with a functional material or a material format as illustrated in FIG. 1. The variety of material formats can enable alternative biosampling approaches that utilize the structural and functional attributes of biopolymer matrices and Key-Cage protein systems. Generally, the material format can be 3D porous matrix, a non-woven membrane, a film, or an ink. In an example, the material format can dissolve in water. In another example, the material format can be only partially soluble in water. The material format can be a film, such as a drop-cast film.

The material format can be a three-dimensional porous matrix having a high surface area-to-volume ratio, such as a sponge or a foam. The material format can be made as a sponge by snap-freezing and lyophilizing solutions containing silk fibroin, Key proteins and Cage proteins. The material format can be a high surface-to-volume porous formats to sample air as a bioresponsive bulk material. For example, the material format can be an air filter.

The material format can be a non-woven membrane, a mesh, or combinations thereof. More specifically, the material format can be an electrospun mat. This material format can serve both for detection and small-pore size filtration by tuning the electrospinning parameters, with end utility in applications such as functional face coverings or air filters for simultaneous protection and monitoring of biological contamination in aerosols.

The material format can be a printable ink. The amphiphilic nature of the protein chains of silk fibroin allows for the generation of pico- to nanoliter solution drops of silk, enabling use in printing technologies. The rheological properties of the inks, such as viscosity and surface tension, do not need to be modified with non-compatible organic or inorganic surfactants, which could negatively interact with doping agents, therefore enabling the functionalization of any substrate of interest.

Printing can provide a modular approach for the use of multiple Key-Cage systems across a variety of surfaces, adding utility to wearable interfaces, surfaces, containers, or other high-touch surfaces providing early warning against exposure to hazardous agents. To generate a printable ink, a solution of silk fibroin can be combined with Key proteins and Cage proteins and additives, for example, Tween. The printable ink can be deposited as spots on a surface, where the spots are arranged singly or printed in a pattern.

Water-based suspensions of silk fibroin can be transformed into a variety of other material formats, including hydrogels, microneedles, and other responsive biointerfaces. These material formats can allow the silk fibroin matrix to be shelf-stable and can be stored without refrigeration while preserving the sensitive detection capabilities of the Key-Cage protein system, as will be discussed later.

### Method of Making the Material Format

The material format can be formed according to the following methods starting from matrix solutions containing Key proteins, Cage proteins, and silk fibroin. In an example, the matrix solution containing Key proteins, Cage proteins, and silk fibroin can be drop-cast and converted into sensitive films after water evaporation. Free-standing films can be formed by casting matrix solutions containing silk fibroin, Key proteins, and Cage proteins on a PDMS substrate and allowing the water to evaporate overnight, leaving a film.

In another example the matrix solution containing Key proteins, Cage proteins, and silk fibroin can be lyophilized to make porous items such as sponges, as shown in FIG. 1. The matrix solution can be lyophilized in such a way that specific desired shapes are formed, for example as spheres or discs. In a further example, printable inks containing Key proteins, Cage proteins, and silk fibroin can be deposited on a substrate by inkjet printing. A device can dispense pico and nanodrops of the matrix solutions containing Key-Cage protein systems and silk fibroin via digitally controlled interfaces on a substrate. The substrate can be a fabric or a polymeric surface. The substrate can be flexible, stretchable, or rigid. As a printable ink, the silk fibroin matrix can be deposited in a variety of printed patterns. The printable ink can be deposited as spots on a substrate, for example, as shown in FIG. 1. The spots can be printed in one or more layers.

In yet another example, matrix solutions containing Key proteins, Cage proteins, and silk fibroin can be electrospun to make non-woven mats. The electrospun mat can be generated by preparing a matrix solution of silk fibroin, Key proteins and Cage proteins, and an additive such as polyethylene glycol, and extruding through a needle having an applied voltage at a flow rate where a grounded fiber collector is positioned away from the needle. The sensing capability of silk fibroin matrix on porous substrates can be improved by fabricating non-woven meshes through direct electrospinning of the silk fibroin matrix.

### Imaging and Sensing Properties

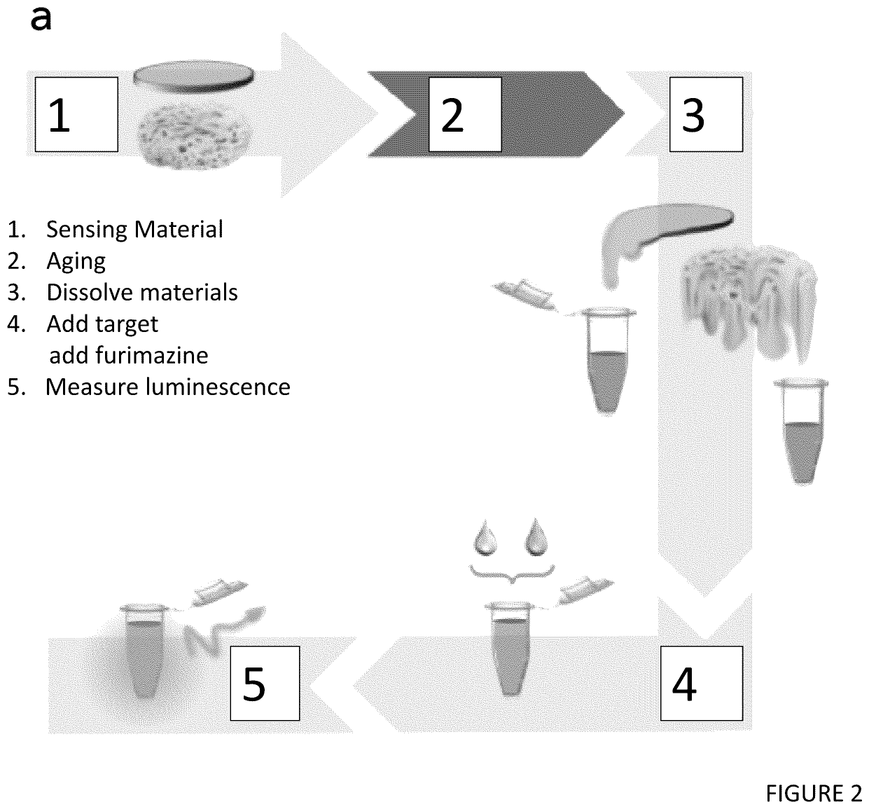

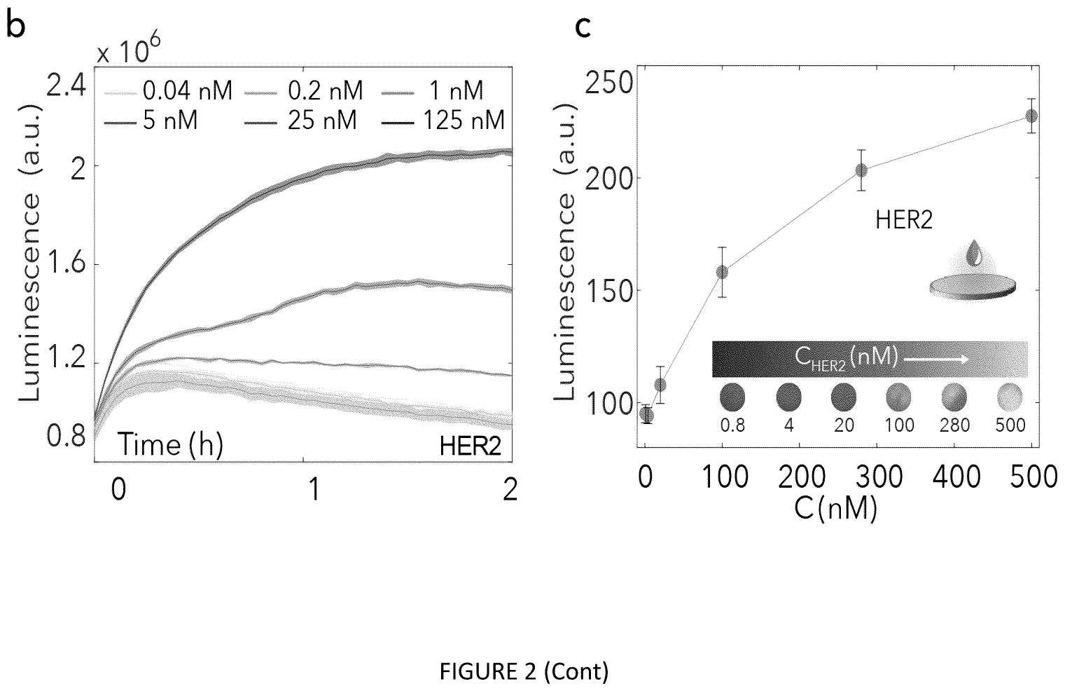

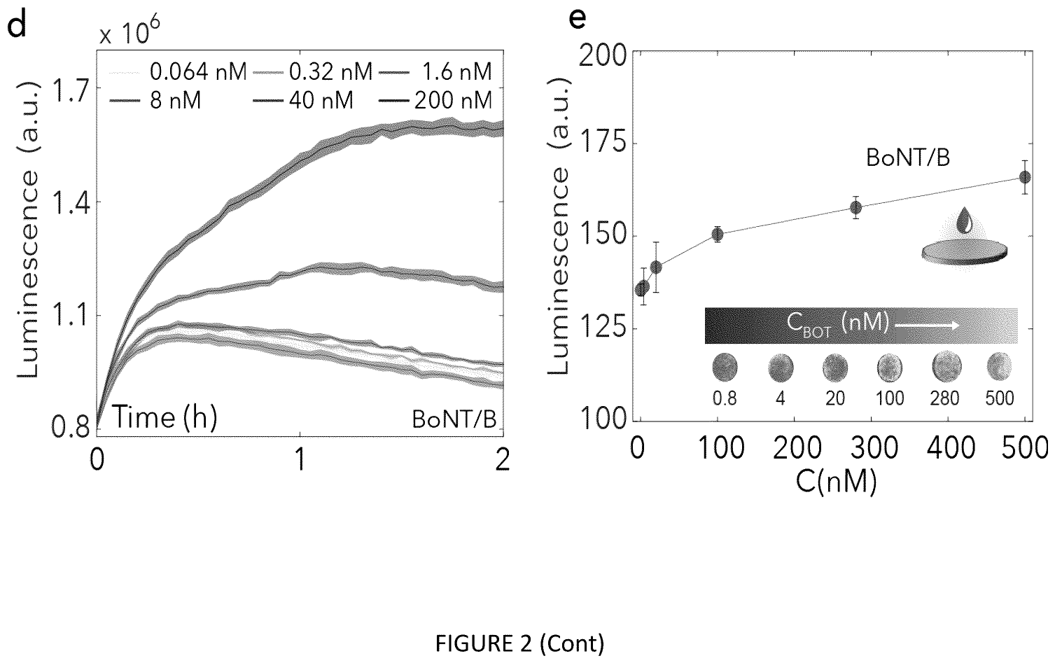

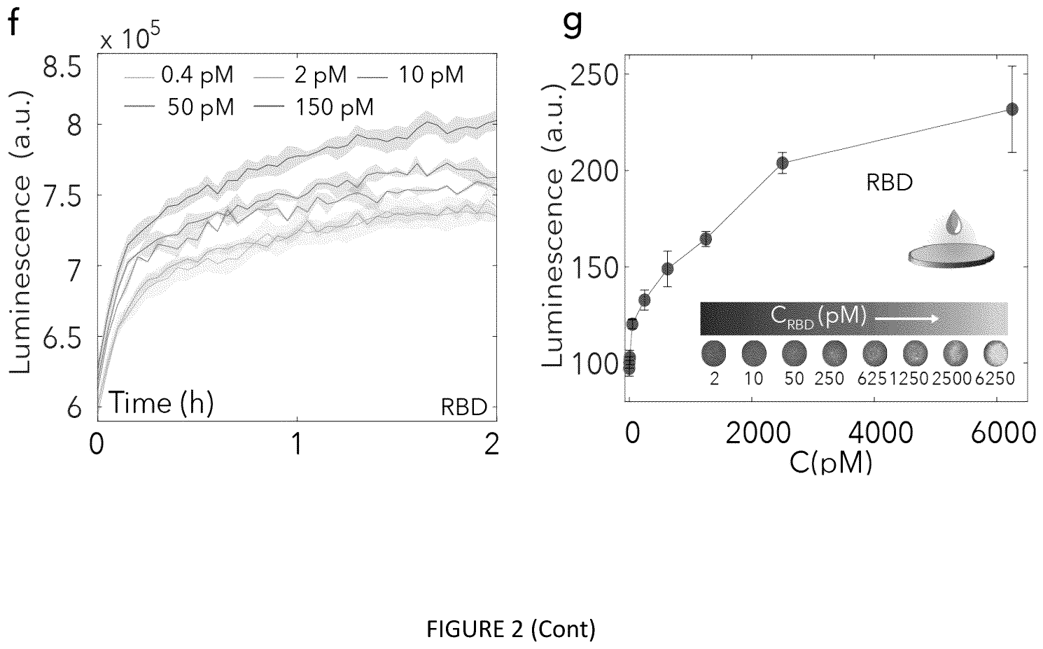

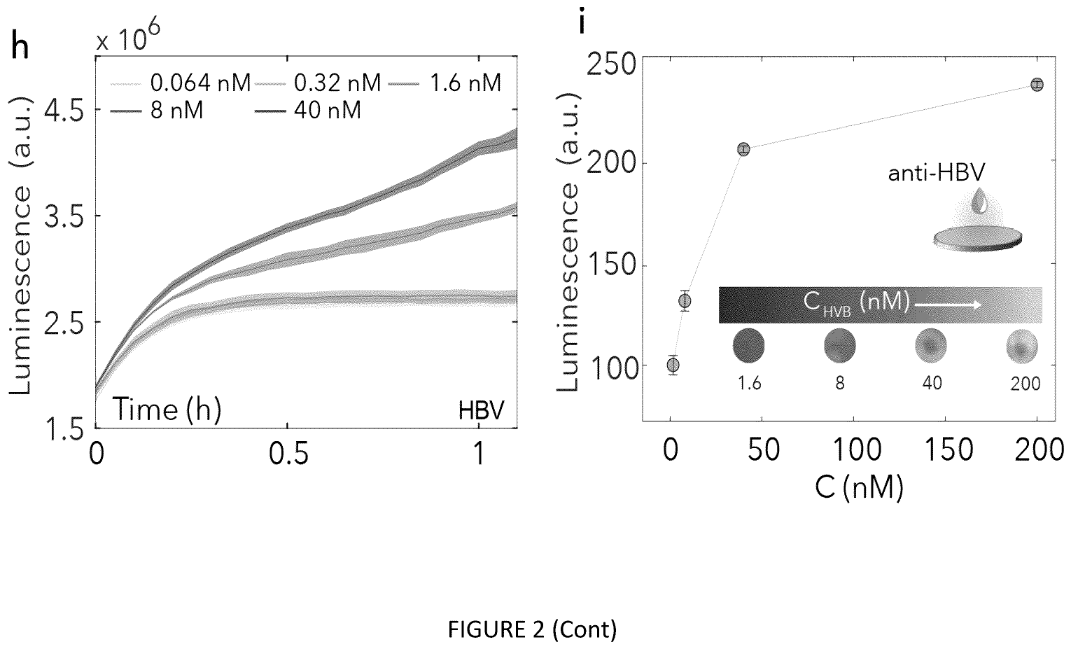

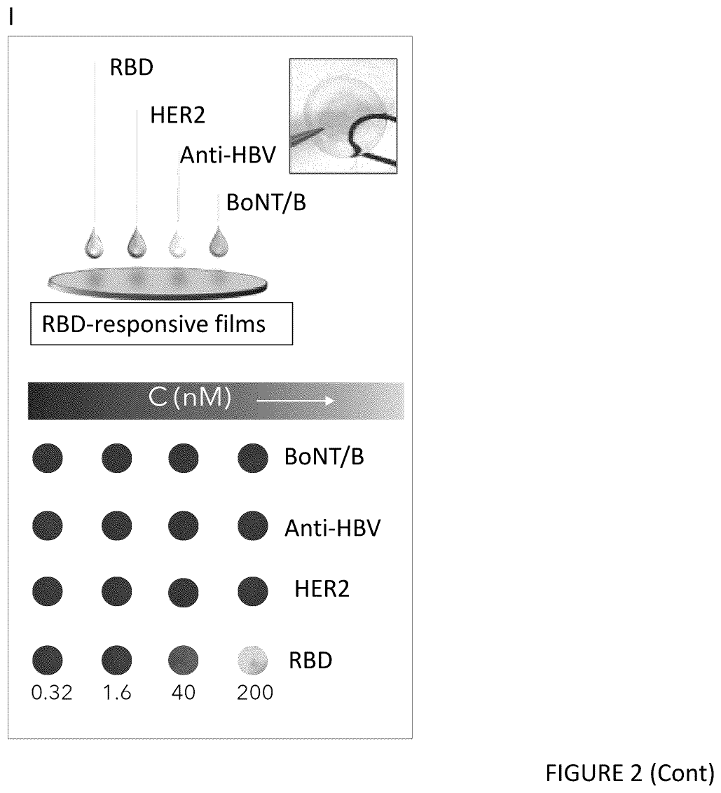

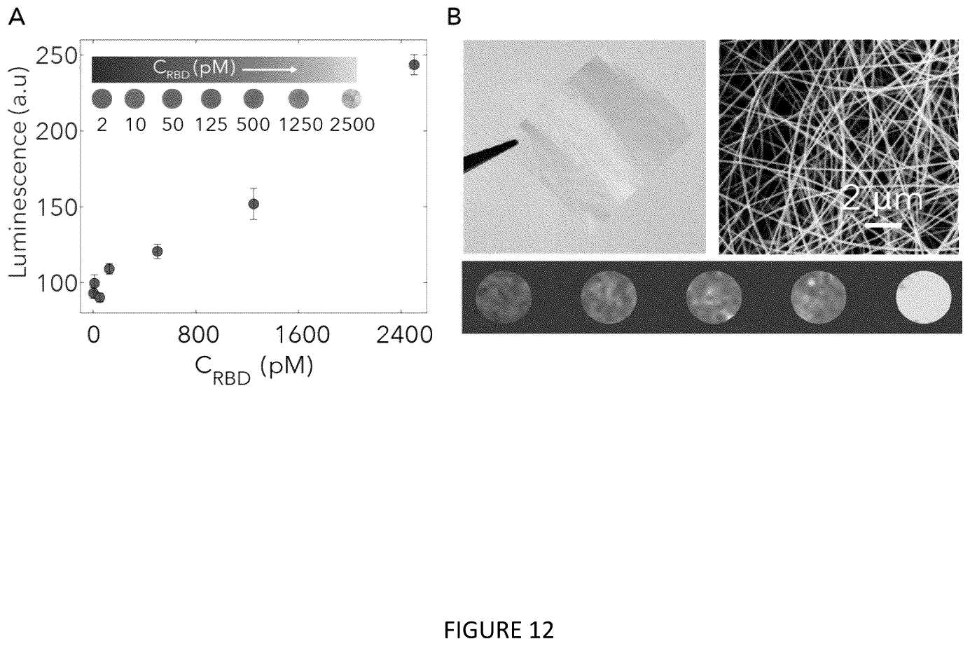

Bioluminescence imaging can be particularly suited for biomarker detection outside laboratory setting given its sensitivity and ease of use. As shown in FIG. 2a, furimazine can be added to a solution having the Key-Cage protein system. Alternatively, a solution of furimazine can be sprayed on an item having the Key-Cage protein system. For example, after electrospinning, the fiber mat was exposed to a target and furimazine before bioluminescence imaging. The results are shown in FIG. 12. Remarkably, in spite of the harsh processing conditions for electrospinning, (in terms of fiber shear forces and high voltages), the sensing performance of the Key-Cage complex can be preserved, covering a 2-1250 pM concentration range.

Addition of furimazine to analyte-bound Key-Cage protein complex results in luminescence, a signal that can be readily observed and measured by a detector. The readout can be easily performed by using low-cost digital cameras and/or smartphones. Although the addition of furimazine is required and the luminescence measurement should be performed in a dark environment, both of these requirements are easily achievable. For example, a kit can be provided that includes furimazine and an app or program that can be integrated with digital cameras or smart phones to facilitate sample processing. The kit can further include an enclosure or covering to assist the user in providing a darkened environment for the readout. Additionally, the kit can include standard solutions for comparison.

When entrained in the silk fibroin matrix, the plurality of Cage proteins and the plurality of Key proteins are not contained, as within a cell, and are not free, as in a solution. When the plurality of Key proteins and the plurality of Cage proteins are entrained in the silk fibroin matrix, the silk fibroin matrix has a silk fibroin matrix sensing property. The silk fibroin matrix sensing property can be a luminescent signal. The intensity of the luminescent signal can be related to the concentration of analyte of interest. The silk fibroin matrix sensing property can be different from one or both of the intracellular sensing property and the solution-phase sensing property. The silk fibroin matrix sensing property can indicate the sensitivity of the Cage-Key protein system to the analyte of interest when the Cage-Key protein system is entrained in the silk fibroin matrix. Remarkably, the silk fibroin matrix sensing property is highly sensitive to the presence of the analyte of interest. This is a surprising result because the mobility of the Key proteins and Cage proteins is decreased when they are entrained in the silk fibroin matrix. This finding enables new sensing applications using new solid form factors (e.g., environmental passive monitoring) that would not be possible with a solution-based system. The de novo proteins are engineered in the lab and are based on a thermodynamic model to allow for their folding/unfolding behavior. It is not obvious that the de novo proteins will maintain their function when they are mixed in a proteinaceous matrix such as silk (i.e. mixing what doesn't exist in nature with what is derived from a “caterpillar”). The folding/unfolding behavior (i.e., the conformational change of the de novo proteins) is an essential feature that allows de novo protein switches to respond to external stimuli (e.g., the presence of markers, toxins, and other contaminants). The preservation of their ability to fold/unfold after the inclusion in a solid silk matrix is the key to the functionality of the sensing system.

### Stability in Matrix

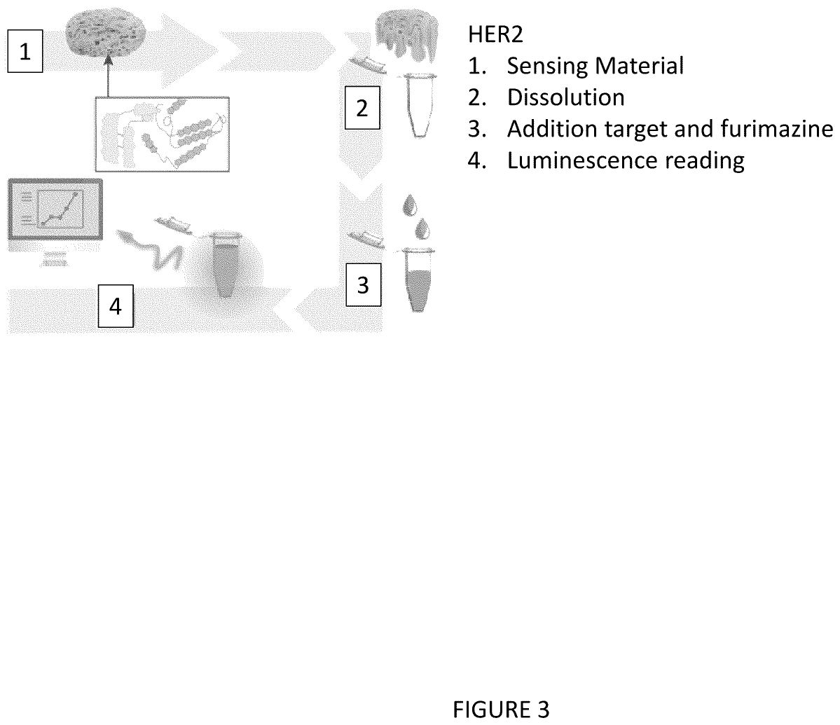

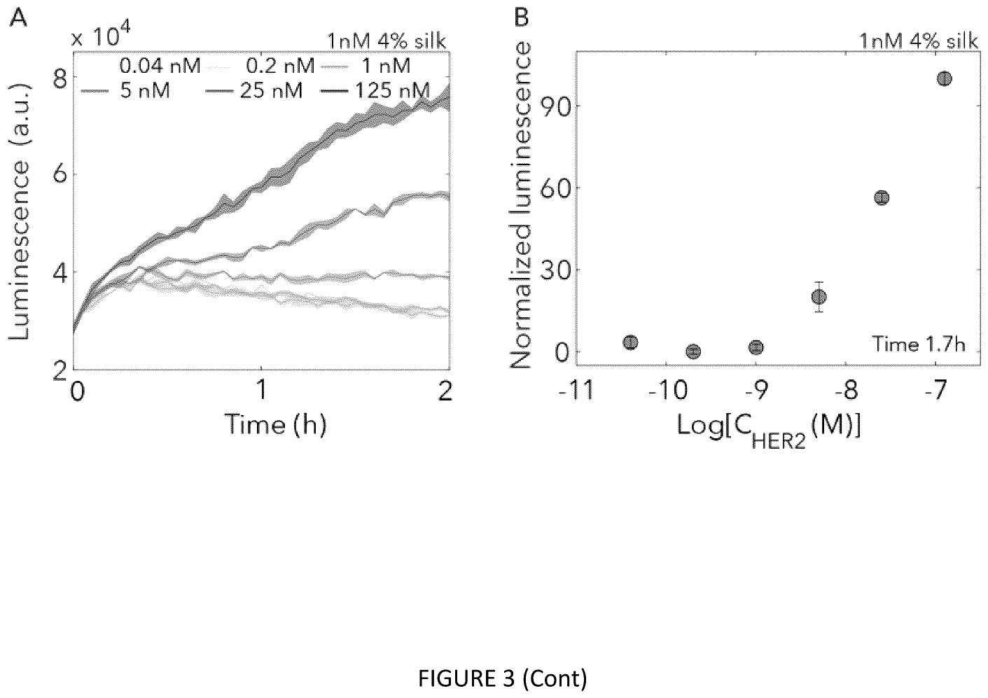

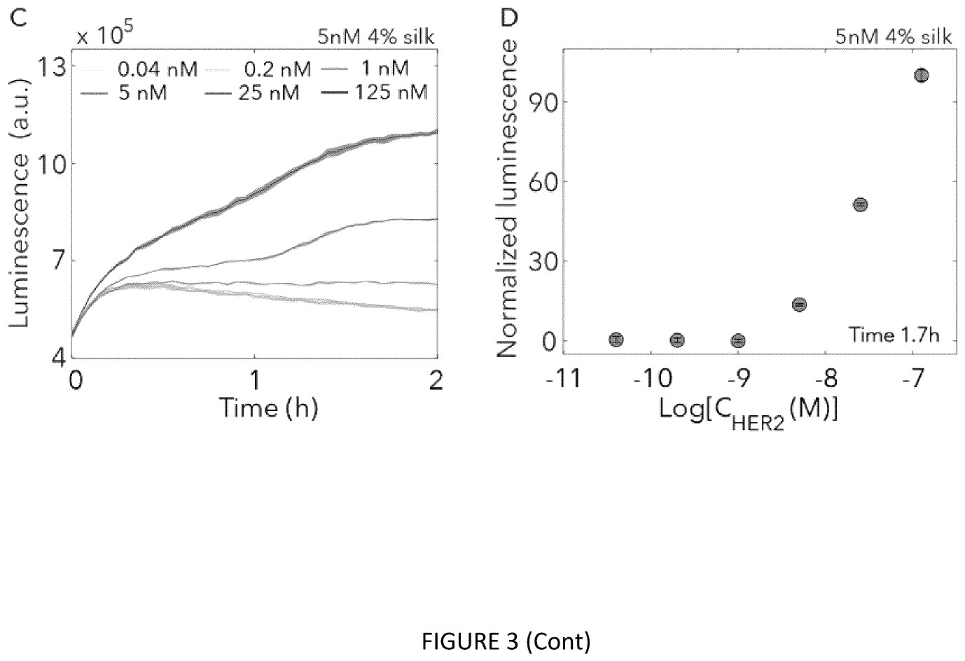

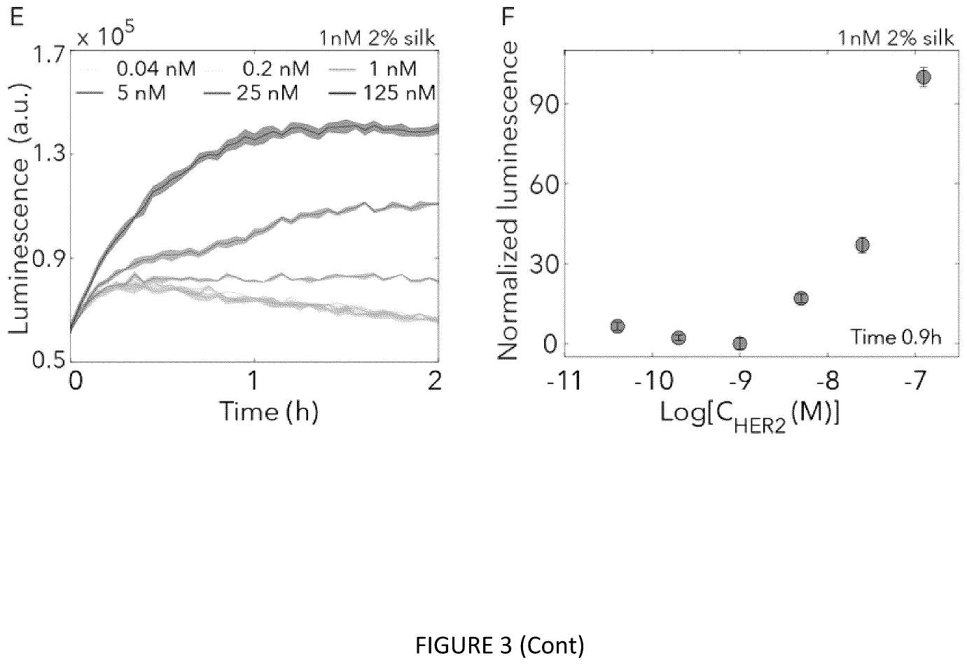

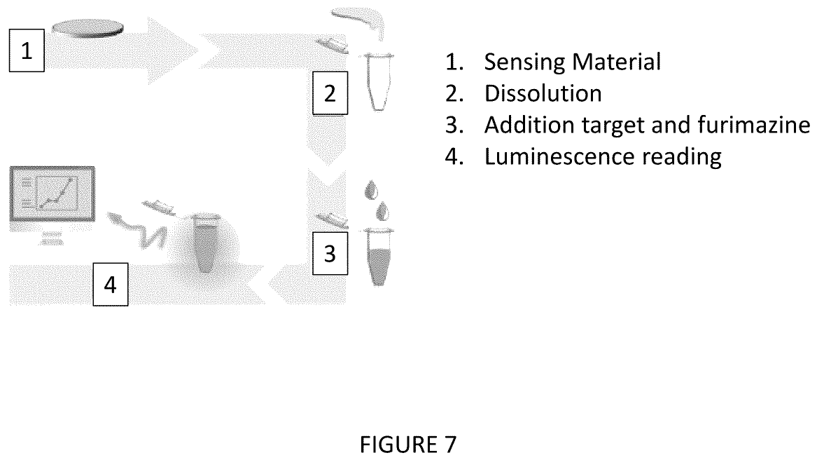

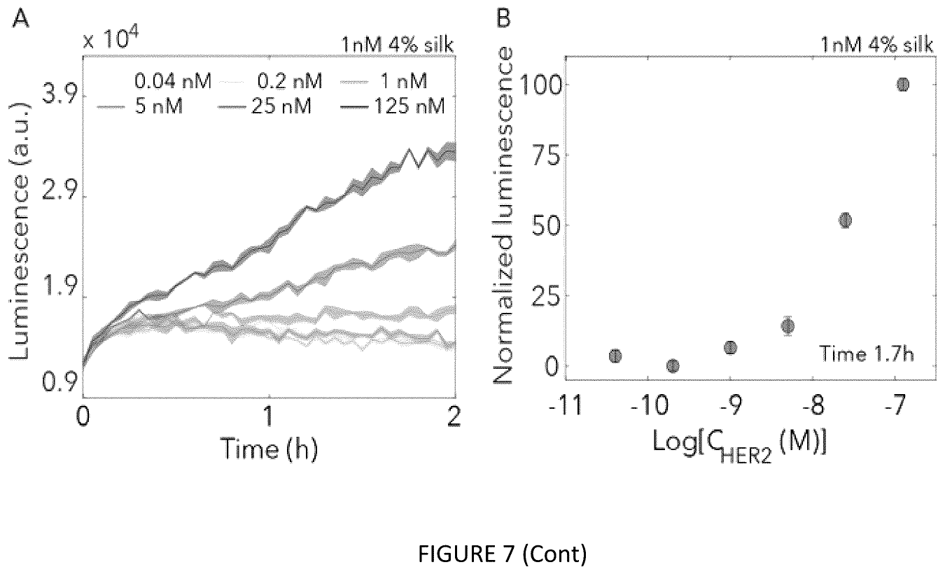

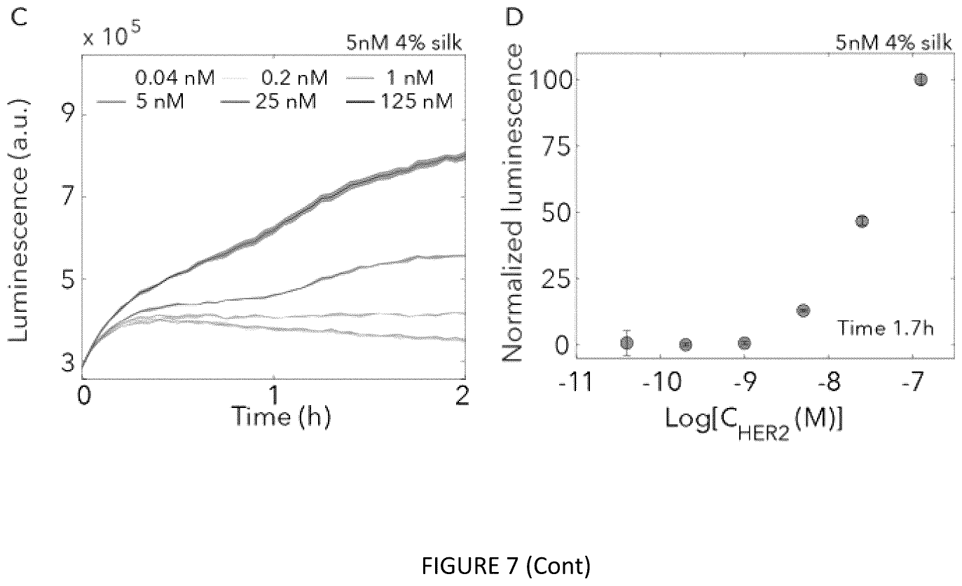

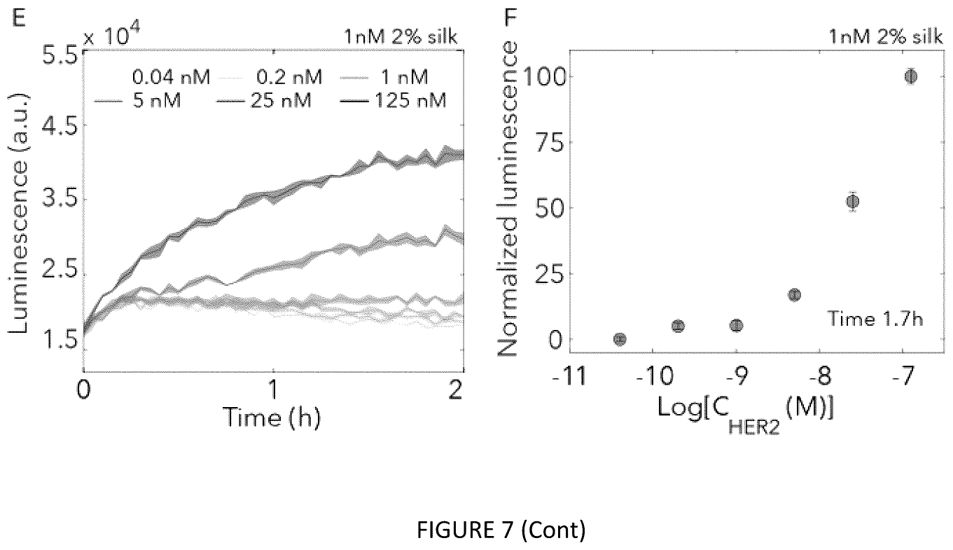

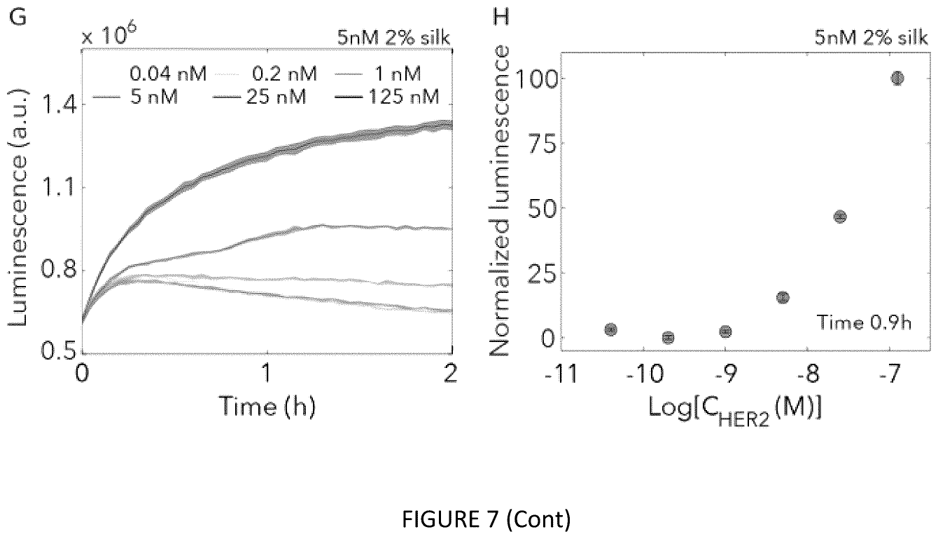

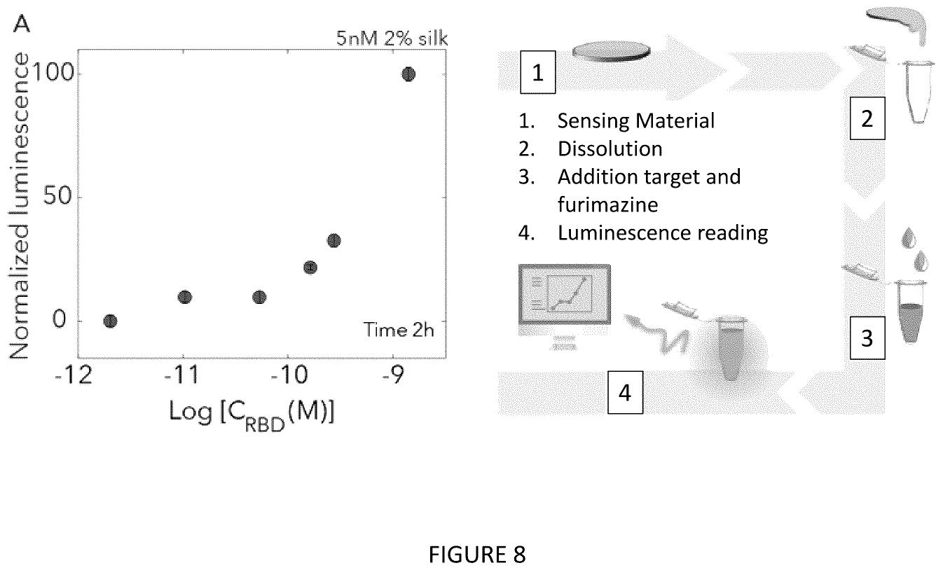

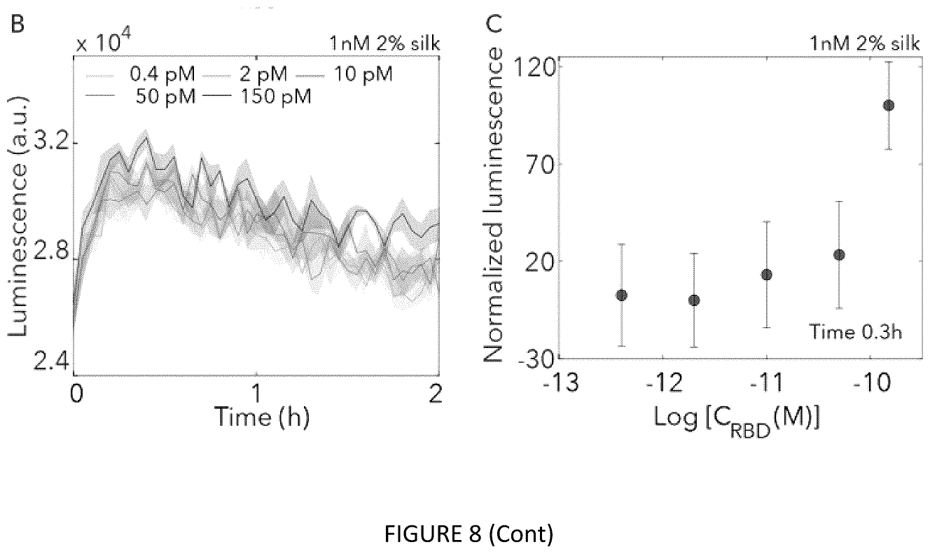

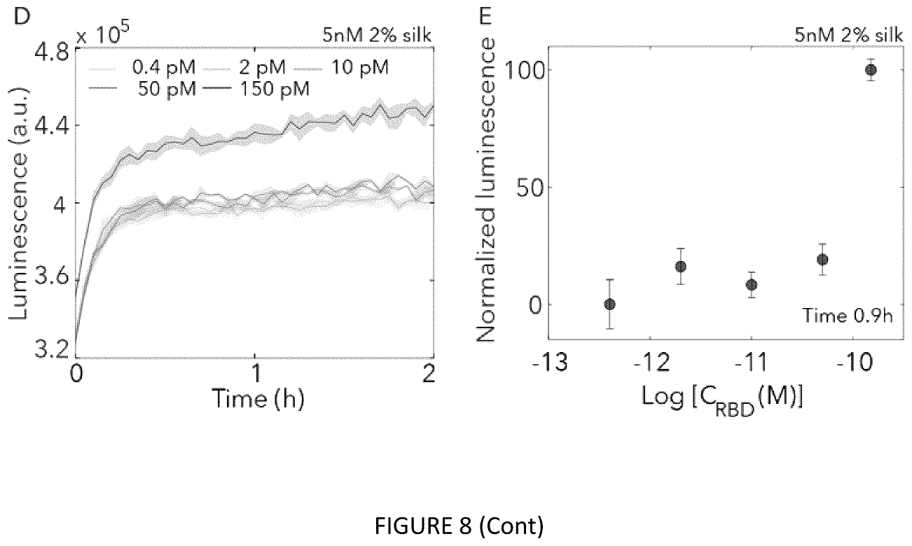

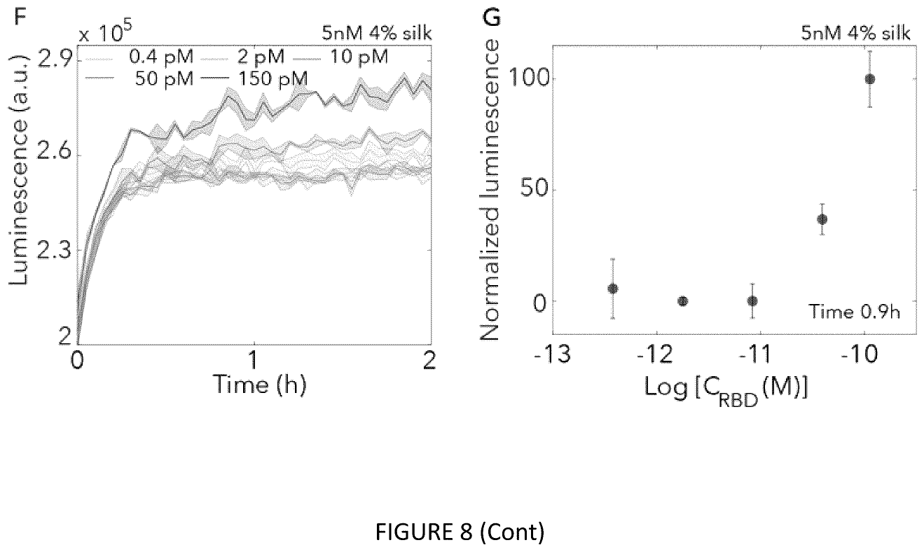

Free-standing bioresponsive films and sponges can be fabricated by adjusting the ratio of Key proteins and Cage proteins within the silk fibroin solution to tune material assembly, performance, and permeability. The viability of the de novo designed Key-Cage protein system contained in the solid material formats can be assessed by dissolving the solid material format, such as sponge or film formats, in water and then exposing them to various concentrations of the analyte of interest to evaluate the protein performance through its chemiluminescent response (FIG. 2). Optionally, the material format can be aged before dissolution and testing. For both sponge (FIGS. 3-6) and film (FIGS. 7-10) material formats, the material format is responsive and generates a luminescent signal that is proportional to increasing concentrations of the analyte of interest, and for all Key-Cage protein systems used, underscoring the versatility and maintained reactivity of the de novo protein-biopolymer composite.

Evaluation of the luminescent signal shows the Key-Cage protein system is responsive and can detect the analyte of interest within minutes (˜10-20 minutes). The Limit of Detection (LOD) for the analyte of interest using can be between nM to pM using reconstituted Key proteins and Cage proteins from material formats such as sponge or film. For example, HER2, BoNT/B, and anti-HBV are detected at nM levels (See FIGS. 2-10). For example, LOD_{HER2sponges}=1 nM, LOD_{HER2films}=1.2 nM; LOD_{BoNT/Bsponges}=2.4 nM, LOD_{BoNT/Bfilm}=2.1 nM; LOD_{anti-HBVsponges}=1.5 nM, LOD_{anti-HBVfilms}=1.8 nM). The Key-Cage protein system can be used to detect RBD down to pM levels (LOD_{sponges}=2 pM, LOD_{films}=11 pM). Thus, the Key-Cage protein system can be used effectively as a protein switch or sensor for analytes even after entrainment in a matrix.

### Solid Form Sensing Article

### Silk Fibroin Matrix Sensing Unit

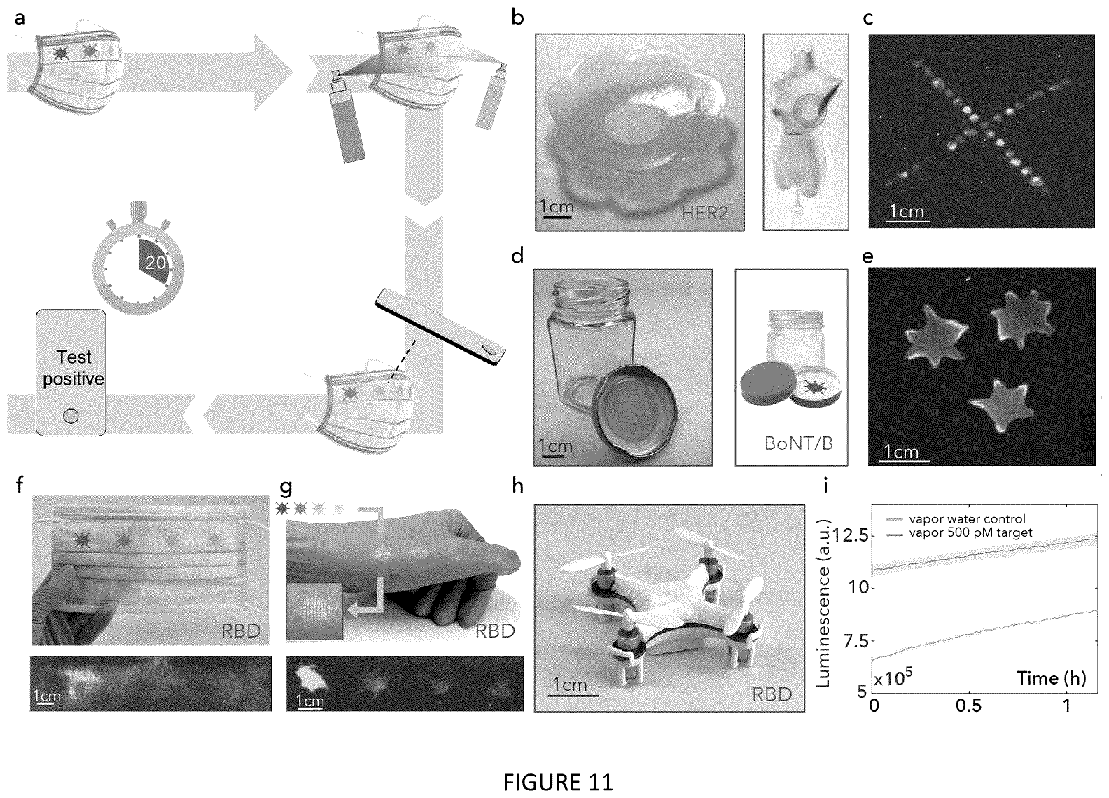

To utilize the sensing ability or analyte responsiveness of the Key-Cage protein system in specific areas, the silk fibroin matrix can be incorporated within a solid form sensing article. Examples of solid form sensing articles include a breast pad, a jar lid, a mask, a glove, and an aerial drone as shown in FIG. 11. The solid form sensing article can include the silk fibroin matrix in any of the above-described material formats, thus including a plurality of Key proteins and a plurality of Cage proteins. The material format can make up the entirety of the solid form sensing article. Alternatively, the material format can make up a portion of the solid form sensing article. More specifically, the material format can be organized as a silk fibroin matrix sensing unit in the solid form sensing article. As in the silk fibroin matrix, the plurality of Cage proteins and the plurality of Key proteins are entrained in the silk fibroin matrix sensing unit.

At least one silk fibroin matrix sensing unit can be incorporated in the solid form sensing article. The silk fibroin matrix sensing unit can make up the entirety of the solid form sensing article. Additionally and alternatively, multiple silk fibroin matrix sensing units can be incorporated in the solid form sensing article. The solid form sensing article can include one or more silk fibroin matrix sensing units. The solid form sensing article can include two, three, four, five, six, seven, eight, nine, or ten or more silk fibroin matrix sensing units. The solid form sensing article can include ten, twenty, thirty, forty, fifty, sixty, seventy, eighty, ninety, or one hundred or more silk fibroin matrix sensing units. The solid form sensing article can have two or more silk fibroin matrix sensing units where at least two of the two or more silk fibroin matrix sensing units have different Cage-Key protein systems tuned to different analytes of interest. The solid form sensing article can have twenty-five, thirty, forty, fifty, sixty, seventy, eighty, ninety, or one hundred or more silk fibroin sensing units, where at least twenty-five, thirty, forty, fifty, sixty, seventy, eighty, ninety, or one hundred have different Cage-Key protein systems tuned to different analytes of interest.

The material format of the silk fibroin matrix and the concentration and distribution of the plurality of Cage proteins and the plurality of Key proteins within the silk fibroin matrix provide a solid form sensing property. When the solid form sensing article is exposed to the analyte of interest, the solid form sensing property of the solid form sensing article is activated. The solid form sensing article can thus be used to detect at least one analyte of interest.

The solid form sensing article can be an item that can be used in or placed in an area where the analyte of interest may be present such as a high-touch surface. In some examples, the solid form sensing article can be a mat, a pin, a sticker, a utensil, or a magnet where the silk fibroin matrix sensing units can be printed on the surface. In other examples, the solid form sensing article can be a type of personal protective equipment such as a glove, a face mask, or an article of clothing. The material format can be a printable ink such that silk fibroin matrix sensing units can be printed on a surface, such as on a glove or mask as shown in FIGS. 1, 11f,g, and 15. The item of personal protective equipment can include at least one silk fibroin matrix sensing unit for detecting an analyte of interest in the environment of the user. Alternatively, the item of personal protective equipment can include at least one silk fibroin matrix sensing unit for detecting an analyte of interest originating from the user.

Additionally, or alternatively, the solid form sensing article can be a free-standing film. The silk fibroin matrix sensing units can be incorporated in the solid form sensing article as solid components. The solid form sensing article can be used for routine monitoring of specific biomarkers, for example with point-of-care devices. In another aspect, the solid form sensing article can be a porous material such as a filter through which air is circulated.

The solid form sensing article can be a personal hygiene item having one or more silk fibroin matrix sensing units. Examples of solid form sensing articles in personal hygiene include disposable wipes, sanitary pads, and dental floss. Generally, the one or more silk fibroin matrix sensing units can be positioned to optimize contact with bodily fluids potentially containing an analyte of interest. In a particular example the solid form sensing article can be a breast pad as shown in FIG. 11b. At least one of the one or more silk fibroin matrix sensing units can be positioned at a nipple-contacting portion of the breast pad, where the nipple-contacting portion contacts a nipple of a user when the breast pad is in use. The at least one of the one or more silk fibroin matrix sensing units can be positioned at an areola-contacting portion, where the areola-receiving portion contacts an areola of a user when the breast pad is in use. The at least one of the one or more silk fibroin matrix sensing units can be positioned at a skin-contacting portion of the breast pad, where the skin-contacting portion contacts a portion of non-nipple, non-areola skin located on a breast of a user when the breast pad is in use.

In another aspect, the solid form sensing article can be a part of an air quality monitoring system for detecting airborne analytes and routine monitoring. In another aspect, the solid form sensing article can be a porous material such as a filter through which air is circulated. In another aspect, the solid form sensing article can be a material exposed to air.

In one example, the solid form sensing article can be incorporated in an aerial drone. The silk fibroin matrix sensing units can be components lodged on the exterior of the aerial drone for exposure to the air during flight. In one example, a viral-sensing aerial drone was generated which was obtained by freeze-drying the silk fibroin matrix in the shape of the top half of the ˜3 cm aircraft (FIG. 11h). The silk fibroin matrix, which is porous, was exposed to the vapors generated by a water bath containing 500 pM RBD target. The analysis of the permeable, structural, bioresponsive material after exposure to RBD, the analyte of interest, exhibited a luminescent response to the analyte of interest, making this approach of utility for standoff environmental sampling and detection of airborne viruses and toxins (FIG. 11i). The porosity of the material format can be tuned such that the bioresponsive lyophilized matrix can allow for enhanced utility in aerosol monitoring in various environments.

In yet another aspect, the solid form sensing article can be a part of a food quality monitoring system for detecting food-borne analytes. The solid form sensing article can be incorporated in a container or a cover for a container. In an example, the silk fibroin matrix sensing units can be printed on the inside of a lid of a jar of food, as shown in FIGS. 11d,e. In another aspect, the solid form sensing article can be a packaging material, such as a plastic film, where the silk fibroin matrix sensing units can be printed on an interior facing side of the film.

The solid form sensing article can be a wearable device or an item of jewelry such as a bracelet, a pin, an earring, or a pendant. The wearable device or item of jewelry can include at least one silk fibroin matrix sensing unit for detecting an analyte of interest in the environment of the user. Alternatively, the wearable device or item of jewelry can include at least one silk fibroin matrix sensing unit for detecting an analyte of interest originating from the user.

### Method of Using the Solid Form Sensing Article

In use, the solid form sensing article can be directly exposed to the analyte. More specifically, the material format including the silk fibroin sensing units can be exposed to the analyte of interest such that the analyte contacts the silk fibroin sensing units. After exposure, the analyte can be lodged on the surface of the material format. The analyte can penetrate the pores of the material format, for example in the case of sponges. The analyte can adhere to fibers, for example in the case of mesh or electrospun mats.

Following exposure to the analyte, at least a portion of the material format containing the silk fibroin sensing units can be dissolved to form an analyte solution. The analyte solution contains silk fibroin, Key proteins, Cage proteins, Key-Cage protein complex, and the analyte. These components are released from the silk fibroin matrix upon dissolution. In the analyte solution, the Key proteins, Cage proteins, and the analyte are mobile and free to form the Key-Cage protein complex with the analyte of interest. The analyte solution is exposed to furimazine such that the Key-Cage protein complex generates a chemiluminescent signal that can be detected.

In one example, the film containing Key proteins and Cage proteins can be partially hydrated with contaminated biological fluids or deionized water that is spiked with the target of interest (mimicking contaminated biological fluids). This step of partial hydration favors the interaction between the analyte and the Key proteins and Cage proteins that are far from the film surface. This approach can provide advantages for use outside the laboratory and does not require complete dissolution of the sensing substrate, which can facilitate usage by untrained users while providing quantitative information on the sensing substrate within minutes.

Additionally, and alternatively, the material format can be used for sensing without complete dissolution of the material format. For example, the material format including the silk fibroin sensing units can be exposed to the analyte of interest, directly exposed to furimazine, and the luminescent signal detected as emanating from the material format. As shown in FIG. 2, HER2 sensing films were exposed to various analyte concentrations and furimazine (FIG. 2c). The resulting luminescent image, analyzed to correlate intensity variations with changes in HER2 exposure, shows a linear response for concentration values between 20-500 nM (LOD=15 nM). Similarly, bioresponsive films were functionalized with Cage proteins and Key proteins as protein switches for BoNT/B (FIG. 2e), RBD (FIG. 2g) and anti-HBV (FIG. 2i), demonstrating the possibility to generate free-standing sensing elements for a variety of targets.

Examples of this method of use are further illustrated in FIG. 11. In these examples, the printed patterns are included in a variety of solid form sensing articles. The sensing units are exposed to the analyte of interest, activated with furimazine, and evaluated for chemiluminescence intensity as a function of detected analyte concentration (FIG. 11a) The analytes of interest that are detected here include HER2 (FIG. 11b-c), Bot/N (FIG. 11d-e), and RBD (SARS-CoV-2, FIG. 11f-g).

In one example the solid form sensing articles are laboratory gloves and surgical masks. In this case the material format is a printable ink, and silk fibroin sensing units sensitive to RBD were printed on the outer surface of the laboratory gloves and exterior surface of a surgical mask. In another embodiment, the silk fibroin sensing units can be printed on the interior surface of a surgical mask. After exposure to RBD, the gloves and mask show a chemiluminescent response proportional to the concentration of the analyte while maintaining the geometry of the printed pattern. The printed patterns provide a luminescent response that is directly proportional to the analyte concentration (FIG. 11, 13). Interestingly, the shear forces and volume ejection that occur during printing do not alter the functional capacity of the printed format, enabling a scalable manufacturing process for the development of ad hoc sensing surfaces.

The surgical mask having sensing units provides a convenient complementary approach to nasopharyngeal swabs for the detection of SARS-COV-2 and was evaluated by spraying furimazine and RBD target to mimic the breathing of potentially infected users (FIGS. 11a, f, 13 A). The absorbing behavior of the mask's non-woven fabric affected the sensitivity of the functional ink. Nonetheless, detected responses show a sensitivity down to concentrations of 0.25 nM on gloves (FIGS. 11g,13B), and 5 nM on the surgical mask.

In another example, the solid form sensing article is a silicone breast pad. This solid form sensing article was selected given the importance of HER-2 as a breast cancer biomarker in nipple fluids and can serve as a potential interface for early detection of breast cancer, to assess the efficacy of treatment, or to discriminate between benign or malignant discharge. HER2 concentration is tracked directly on the dotted pattern dispensed on the silicone pad in a short timeframe (˜20 minutes) and within the nM range (FIGS. 2c, 11b-c, 13C). Sensing breast pads could represent an alternative avenue for early detection of breast cancer when spontaneous nipple discharge is present. Furthermore, as de novo protein switches capable of targeting different cancer biomarkers with high sensitivity and specificity can be also developed, this strategy opens the path for non-invasive detection and monitoring of human cancer through biomarkers present in biological fluids (e.g., salivary proteins for detection of oral, head, or neck cancers).

In yet another example, the detection of toxins relevant to food safety and human health such as botulin were also explored by using relevant de novo Key-Cage protein systems in a matrix of silk fibroin as a printable ink and printed as patterns on the inside of a food jar lid. As in the case described above, this approach could allow for the detection of contamination, spoilage, or tampering of the contents by examination of the lid's luminescence response in 20 minutes and at the toxin concentration within the nM range (FIGS. 2e, 11d,e, and 13D). The detection of botulin directly on a contaminated food container is particularly useful, given the high toxicity of botulinum neurotoxin (which is lethal to babies at a dose of ng/kg) and that many outbreaks of botulinum are caused by improper food handling practices with homemade food.

### Interference and Specificity

Biosensors are prone to interference given the large amounts of non-specific interactions with background molecules, which can affect their sensing capabilities in biofluids. To further evaluate the response of the silk fibroin matrix sensing units to different specimens, simulated saliva spiked with different concentrations of RBD was used to test the performance of a solid form sensing article (a responsive film) in complex media. RBD was detected in simulated saliva down to 10 pM concentrations without compromising the dynamic sensing range (FIG. 2g).

Additionally, the specificity of RBD bioresponsive films was evaluated by testing their responsiveness to potentially interfering analytes (FIG. 2l). RBD films were exposed to furimazine and to different targets (RBD, HER2, BoNT/B, and HBV) at various concentrations in the nM range (0.32-200 nM) so that each RBD film was exposed to only one target. After exposure, the RBD films were analyzed via bioluminescence imaging and were found to be active and responsive only to the desired target (i.e., RBD protein) demonstrating the specificity of the silk fibroin matrix sensing units.

### Accelerated Aging

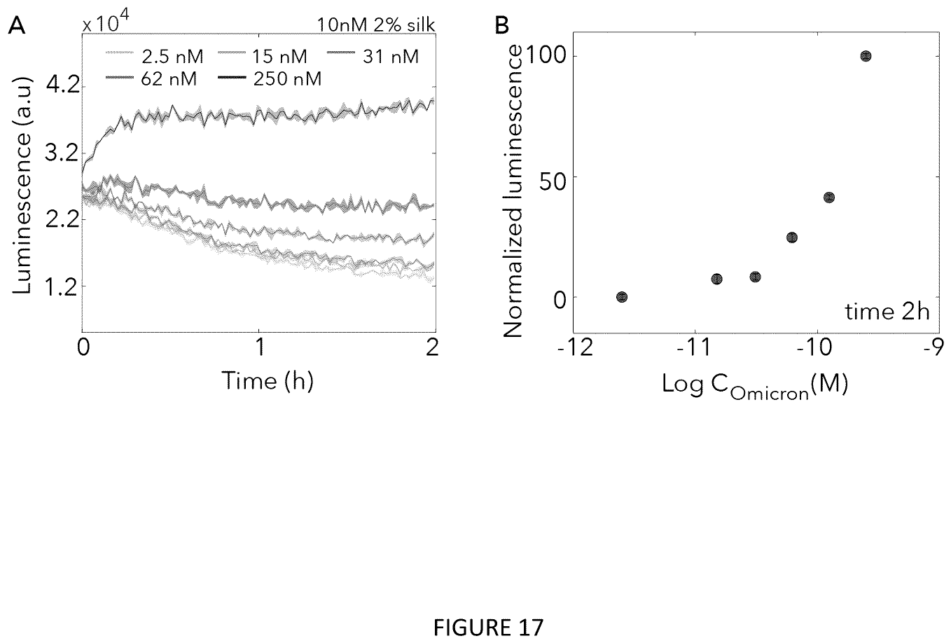

Although the Key-Cage protein systems can be stored in refrigerated conditions at −80° C., the integration in the silk fibroin matrix can preserve the sensing performance (FIG. 17) for long periods of time. The sensing performance can be characterized by one or both of sensitivity and dynamic range. The solid form sensing article can maintain a solid form sensing performance after storage under a range of conditions. In one example, the solid form sensing performance can be maintained after storing the solid form sensing article at standard ambience (i.e., temperature of 25° C. and air humidity in the range of 25-40%) for one year. In another example, the solid form sensing performance can be maintained after storing the solid form sensing article at extreme heat (i.e., temperature of 60° C., up to 4 months). The sensing performance can be at least 90%, at least 95%, at least 99%, or 100% of the original sensing performance before storage.

### Advantages

De novo designed protein switches such as the Key-Cage protein system are powerful tools to specifically and sensitively detect diverse targets with simple luminescent readouts. Finding an appropriate material host for de novo design protein switches without altering their thermodynamics while preserving their intrinsic stability over time enables the development of a variety of sensing formats to monitor exposure to pathogens, toxins, and for disease diagnosis. Silk fibroin is an attractive matrix for lyophilization to obtain stable silk fibroin matrices that can be quickly (within seconds) dissolved for readout. Additionally, silk provides material versatility, including the ability to cast films which reduces the cost of fabrication and processing time and results in shelf-stable formats that can be easily handled and shipped for analysis in the field.

Silk fibroin is advantageous because of its polymorphism, which allows for processing and reshaping into different material composites that preserve the biological function of their constituents.

The demonstrations included herein show the interplay between form and function, where the different bioresponsive formats can be directed to different end uses. The different formats can be tuned for optimal analyte diffusion and permeability by control of the directed assembly of the silk fibroin matrix through the modulation of its inter- to intramolecular bond ratios for efficient analyte collection, sampling, and sensing. This versatility ultimately can be exploited to optimize sensor performance and use in a variety of settings, from the laboratory to the clinic, to the home.

Disclosed herein is a de novo protein-biopolymer hybrid that maintains the detection capabilities induced by the conformational change of the incorporated proteins in response to analytes of interest in multiple, shelf-stable material formats without the need of refrigerated storage conditions. A set of functional demonstrator devices including personal protective equipment such as masks and laboratory gloves, free-standing films, air quality monitors, and wearable devices is presented to illustrate the versatility of the approach. Such formats are designed to be responsive to human epidermal growth factor receptor (HER2), anti-hepatitis B (HBV) antibodies, Botulinum neurotoxin B (BoNT/B), and severe acute respiratory syndrome coronavirus 2 (SARS-CoV-2). This combination of form and function offers wide opportunities for ubiquitous sensing in multiple environments by enabling a large class of bio-responsive interfaces of broad utility.

### MISCELLANEOUS

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected. For example, any of the features or functions of any of the embodiments disclosed herein may be incorporated into any of the other embodiments disclosed herein.

### Examples

### Materials

Sodium carbonate, lithium bromide, and 1× phosphate buffer (PBS) were purchased from Sigma-Aldrich (USA). Ethanol (100%) and Teknova 10% TWEEN-20 solution were purchased from Fisher Scientific. The furimazine substrate was part of the kit Nano-Glow® Luciferase Assay System and was purchased from Promega. Human HER2/ErbB2 Protein, His Tag (MALS verified) was purchased from Acro Biosystems. LucKey and lucCage constructs (lucCageRBD, lucCageHer2, lucCageHBVa and lucCageBot) were expressed and purified as previously reported (See: Tao, H. et al. Inkjet Printing of Regenerated Silk Fibroin: From Printable Forms to Printable Functions. Adv Mater 27, 4273-4279 (2015). https://doi.org:10.1002/adma.201501425, which is hereby incorporated by reference in its entirety for all purposes). Monomeric SARS-CoV-2 RBD was expressed and purified as previously described (See: Marelli, B. et al. Programming function into mechanical forms by directed assembly of silk bulk materials. Proc Natl Acad Sci USA 114, 451-456 (2017). https://doi.org:10.1073/pnas.1612063114, which is hereby incorporated by reference in its entirety for all purposes). The anti-HBV antibody HzKR127-3.2 was purified as previously described (See: Langan, R. A. et al. De novo design of bioactive protein switches. Nature 572, 205-210 (2019). https://doi.org:10.1038/s41586-019-1432-8; and Quijano-Rubio, A. et al. De novo design of modular and tunable protein biosensors. Nature 591, 482-487 (2021), both of which are hereby incorporated by reference in their entirety for all purposes). Botulinum neurotoxin B HcB expressed as previously reported (See: Matzeu, G. et al. Large-Scale Patterning of Reactive Surfaces for Wearable and Environmentally Deployable Sensors. Adv Mater 32, e2001258 (2020). https://doi.org:10.1002/adma.202001258, which is hereby incorporated by reference in its entirety for all purposes). All chemicals were used as received and they followed trace metal standard, when possible. Silk cocoons from Bombyx Mori silkworm were purchased from Tajima Shoji Co. (Yokohama, Japan). Deionized water with resistivity of 18.2 MΩ cm was obtained with a Milli-Q reagent-grade water system and used to prepare aqueous solutions. Polystyrene sheets (1.6 mm thickness) used as substrates for printing were purchased from McMaster Carr. 96 well plates (Corning® 96 Well Half-Area Microplate, CLS3693) were purchased from Sigma-Aldrich (USA). Surgical masks (Disposable 3-Ply Masks, 12-888-001A), laboratory nitrile gloves (579585XS), and PCR tubes were purchased from Fisher Scientific. Simulated saliva (1700-0305) was purchased from Pickering Laboratories.

### Methods

### 1. Silk Fibroin Isolation

Regenerated silk fibroin (RSF) solution was isolated from B. mori cocoons as previously reported (Langan, 2019; Quijano-Rubio, 2021). Briefly, silk cocoons were shredded, separated from impurities, and boiled in a 0.02 M solution of sodium carbonate to remove sericin. Boiling time was set to 120 minutes to fabricate bio-reactive films, sponges, and inks, and to 30 minutes to prepare mixes for electrospinning. The fibers were rinsed with deionized water three times for 20 minutes and air-dried overnight. The dried silk mats were subsequently dissolved in a 9.3 M solution of lithium bromide at 60° C. for 4 hours. The chaotropic salt was then removed through dialysis (dialysis membranes MWCO 35 kDa) against deionized water for 48 hours, changing the water 6 times at regular intervals, which yielded a 7-8 wt % silk solution. The final solution was centrifuged at ˜12,700×g for 20 minutes, filtered to separate remaining impurities, and stored at 4° C.

### 2. Fabrication of Silk-Based Sponges and Films

LucCage and lucKey of desired concentrations were prepared from the initial stock solutions listed in Table 1. Aliquots of the lucCage and lucKey stock solutions were combined with silk solutions at 2 wt % or 4 wt %, to obtain sensing films and sponges with different concentrations, as reported in Table 2. To prepare bio-reactive film formats, 100 μL of the sensing mix was cast on PDMS substrates. Films were dried at room temperature (25° C.) overnight. To prepare bio-reactive sponges, 100 μL of silk-based solution was aliquoted in PCR tubes, flash-frozen, and lyophilized overnight with a Labconco FreeZone 2.5 Liter Benchtop Freeze Dry System (USA). Bio-reactive films and sponges were stored in Eppendorf tubes at room temperature before undergoing further testing.

TABLE 1
Initial concentration of stock solutions.

[Concentration] (μM)

| | Key | Cage |
|---|---|---|
| RBD | 91 | 29.5 |
| HER2 | 61 | 37 |
| BoNT/B | 91 | 100 |
| Anti-HBV | 91 | 47.5 |

TABLE 2
Composition of sponges and films.

| Sample format | Sponges | Film |
|---|---|---|
| Sample volume (μL) | 100 | 100 |
| [Silk] | 2% and 4% | 2% and 4% |

| Analytes | HER2 | RBD | BoNT/B | Anti-HBV | HER2 | RBD | BoNT/B | Anti-HBV |
|---|---|---|---|---|---|---|---|---|
| [Analytes] (nM) | 1 | 1 | 25 | 6.25 | 1 | 1 | 25 | 6.25 |
| | 5 | 5 | | | 5 | 5 | 50 | 25 |
| | | | | | 25 | 10 | | |
| | | | | | | 15 | | |
| | | | | | | 20 | | |
| | | | | | | 25 | | |

### 3. Preparation of Silk-Based Inks

The lucKey-lucCageRBD stock solutions were diluted to the final concentration of 25 nM in 2 wt % silk fibroin solution using deionized water prefiltered with a 0.22 m syringe filter. The solution was mixed with a plate shaker for 45 minutes to enable pre-equilibration and subsequently 0.2 wt % Tween 20 was added to yield the printing mix. 20 μL of silk ink was printed onto white polystyrene substrates, surgical masks, and nitrile laboratory gloves using an HP D300e Digital Dispenser and T8+ dispense head cassettes. The printing process was influenced by the viscosity of the solution, the surface tension, and the alignment between substrates and dispense head, which affected the dispensing accuracy. Furthermore, the hydrophobicity of the substrates greatly impacted the resolution of the printed patterns. Although different types of surfaces were used, the thickness of the chosen substrates (1.6 mm) enabled high dispensation accuracy and spot alignment during the printing process. The printed patterns were designed with the HP Bio Pattern software. The printing resolution was optimized by tuning the number of layers dispensed and the volumes dispensed per-spot. The final volume of the pattern was given by the total volume dispensed per-spot multiplied by the number of spots in the pattern. 25 nM LucKey-lucCageHER2 and 50 nM BoNT/B solutions were prepared and manually dispensed on a silicone breast pad in 2 ul drops (HER2) or a jar lid in 20 ul (BoNT/B).

### 4. Bioluminescence Kinetics of Films and Sponges

Chemiluminescent readings were recorded with a Synergy HT Microplate Reader (BioTek, USA). The sensing silk-based sponges and films were fully dissolved in deionized water and mixed for 45 minutes before analysis. 15 μL of furimazine substrate (Nano-Glo luciferase assay reagent, Promega) was added to the sensing mix to obtain a final total volume of 2015 μL. 95 μL aliquots of the lucKey-lucCage silk solutions were transferred into a white polystyrene 96 well plate and the solution was monitored to obtain the baseline reading by acquiring the bioluminescence signal every 3 minutes for 50 minutes. After this time, 5 μL of serially diluted target proteins were added to the mix into the 96 well plate. The final solution was mixed for 5 minutes. Bioluminescence kinetics were recorded for 3 additional hours to acquire the response of the dissolved bio-reactive silk formats in presence of various analytes concentrations. The limit of detection (LOD) was calculated as the value related to the cross point between the extrapolation of the lines defining the nonresponsive range and linear-response range of the system.

### Supplementary Methods

### 1. Biosensor Protein Production and Purification

The lucCage biosensor proteins lucCageRBD, lucCageHBVa, lucCageHER2, lucCageBot and lucKey were purified as described. Briefly, glycerol stocks of E. coli Lemo21(DE3) cells (NEB) previously transformed with pET29b+ plasmids encoding the synthesized genes of interest were grown for 24 hours in LB media supplemented with kanamycin. The starter culture was then inoculated at a 1:50 mL ratio in the Studier TBM-5052 autoinduction media supplemented with kanamycin, grown at 37° C. for 2-4 hours, and then grown at 18° C. for an additional 18 h. Cells were harvested by centrifugation at 4000 g at 4° C. for 15 min and resuspended in 30 ml lysis buffer (20 mM Tris-HCl pH 8.0, 300 mM NaCl, 30 mM imidazole, 1 mM PMSF, 0.02 mg/mL DNAse). Cell resuspensions were lysed by sonication for 2.5 minutes (5 second cycles). Lysates were clarified by centrifugation at 24,000 g at 4° C. for 20 min and passed through 2 ml of Ni-NTA nickel resin (Qiagen, 30250) pre-equilibrated with wash buffer, (20 mM Tris-HCl pH 8.0, 300 mM NaCl, 30 mM imidazole). The resin was washed twice with 10 column volumes (CV) of wash buffer, and then eluted with 3 CV of elution buffer (20 mM Tris-HCl pH 8.0, 300 mM NaCl, 300 mM imidazole). The eluted proteins were concentrated using Ultra-15 Centrifugal Filter Units (Amicon) and further purified by using a Superdex™ 200 Increase 10/300 GL (GE Healthcare) size exclusion column in Tris Buffered Saline (TBS; 25 mM Tris-HCl pH 8.0, 150 mM NaCl). Fractions containing monomeric protein were pooled, concentrated, snap-frozen in liquid nitrogen, and stored at −80° C.

### 2. Bioluminescence Imaging

Bioluminescence images were acquired with the Molecular Imaginer® ChemiDoc XRS+Imaging System (Bio-Rad) using the Chemi High Resolution mode. Images were acquired at time intervals of 5 minutes, for a total of 25 minutes.

Imaging of films: For these experiments, RSF films were prepared with the same mix used for printing. Briefly, 20 μL of bio-reactive silk solution at a final concentration of 2 wt % were cast on polystyrene substrates and let dry overnight at room temperature. To analyze the film responsiveness to various target concentrations, films were dissolved in 15 μL of deionized water containing 3% of furimazine substrate. For the experiments with RBD, films were dissolved both in water and in simulated saliva before readouts. After the addition of furimazine, 5 μL of serially diluted target solutions were added to the dissolved films to evaluate the bioluminescence signal for each analyte concentration. Example results are shown in FIG. 2.

To check the specificity of the sensing films, interference experiments were carried out using lucCageRBD films. These were exposed to 5 μL of serially diluted RBD target (control) or interfering analytes (BoNT/B, anti-HBV antibody, and HER2 ectodomain protein), and the corresponding bioluminescence signal was detected. The concentration of the serial dilutions of the analytes was set to 0.32, 1.6, 40 and 200 nM. The results are shown in FIG. 2l.

Imaging of patterned ink (FIGS. 11g,c,e): The same procedure described above was used to test the bioluminescence signals of the silk-based patterns dispensed on laboratory gloves, the jar lid (as viral particle sketches), and on the silicone breast pad. To maintain the lucKey-lucCage concentration constant, the volume of water added to each printing design was adjusted accordingly.

Imaging of surgical masks (FIG. 11f): The surgical masks were tested mimicking a real scenario to simulate the use of non-specialized users. In this case, the furimazine substrate solution (2.5 wt %) and target protein solutions (30 nM and 1 nM) were sprayed on the mask, without precise control on the procedure (in terms of sprayed volumes and area of the mask), and the bioluminescence signal was monitored for 25 minutes as previously discussed.

For an easier visualization, all the collected images were processed in false color (reverse Viridis color map reference) and using image processing libraries in Python software. The reverse Viridis color map associates high luminesce readings to yellow hues and low/absent signals to blue hues. The green gradient that developed between the blue and yellow hues accounts for the growth in chemiluminescence readouts. The false color images were post-analyzed using the software Fiji-ImageJ to quantify and relate the intensity variations in luminescence to variations in analyte concentrations.

### 3. Preparation of Silk-Based Electrospun Non-Woven Mats

The lucKey-lucCageRBD initial stock solutions were diluted in deionized water and mixed for 45 minutes with a plate shaker to enable equilibration. The solution was then combined with a mixture of 7 wt % RSF and 1.2 wt % polyethylene glycol to obtain a final concentration of lucKey-lucCageRBD of 25 nM. A custom-made electrospinning setup was used to fabricate the non-woven mats. Prior to electrospinning, the metal collection circular plate was coated with Teflon spray to facilitate sample removal after electrospinning. 1 mL of solution was then extruded through an 18-gauge needle at a flow rate of 0.90 mL/hr. An applied voltage of 16 kV to the needle caused jet initiation and fiber collection on a grounded collector positioned 18.5 cm away. The electrospinning apparatus was contained in an electrically insulated and environmentally controlled enclosure which was maintained at 25% humidity. After electrospinning, the fiber mat was collected, cut in circles (1 cm of diameter) with a laser cutter (Trotec Speedy 300) and subsequently exposed to target and furimazine before bioluminescence imaging. Results are shown in FIG. 12.

### 4. Responsiveness of RBD Sponges to RBD-Spiked Vapor

To test the ability of the bio-responsive silk sponges to detect the SARS-CoV-2 RBD protein in air, 25 nM lucKey-lucCageRBD silk sponges were exposed to aqueous vapor with and without 500 pM of RBD target protein. The sponges were placed in a stainless-steel cylindrical chamber kept on a heating plate at 120° C. containing a water bath to generate the water vapor and exposed for 20 minutes. To prevent the direct contact of the sponges with the bath, they were placed on a metallic mesh. After the exposure to the vapor, the bioluminescence response of the sponges dissolved in deionized water was acquired for 1 h with a bench top plate reader.

### Supplementary Text

### Screening of Silk-Based Solutions for HER2 Detection

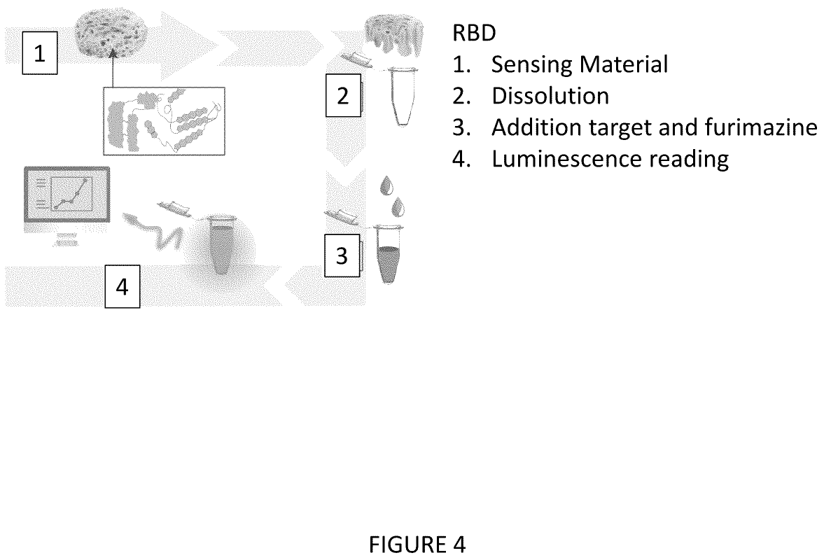

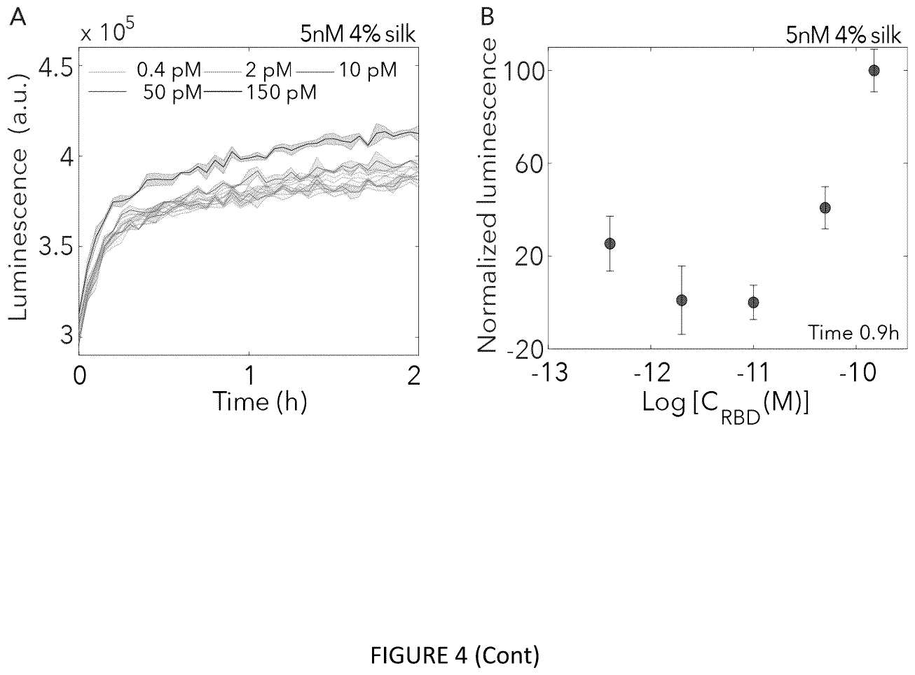

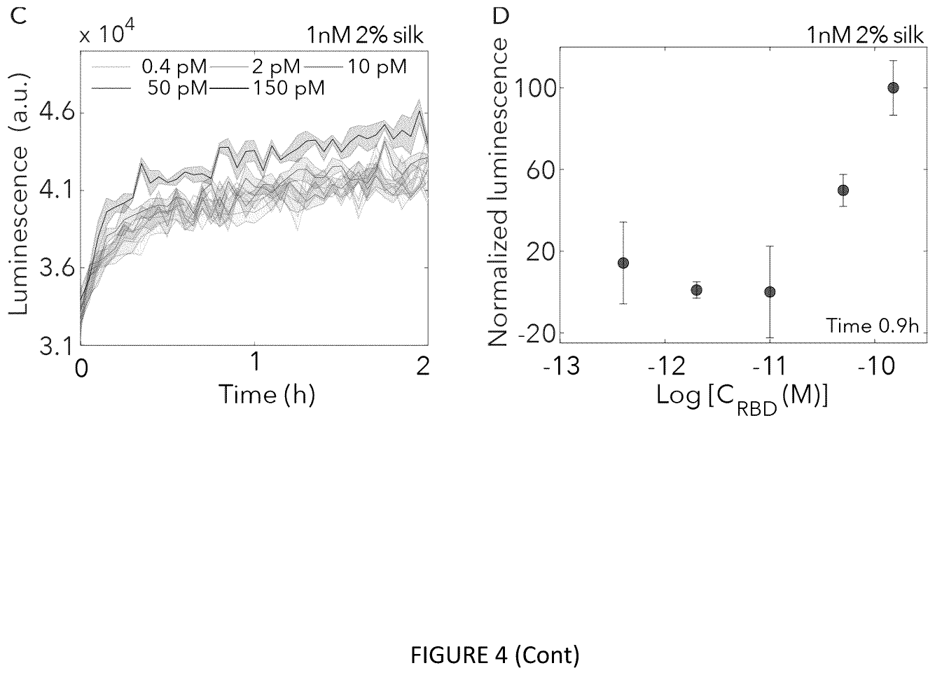

Preliminary studies were carried out to assess the impact of RSF at different concentrations on the lucKey-lucCage protein biosensor functionality and the HER2 protein complex was used as a model. In these experiments, different combinations of lucKey-lucCage at final concentrations of 1 nM and 5 nM were embedded into RSF solutions at 4 wt %, 3 wt %, 2.5 wt %, and 2 wt % (FIG. 4). These bio-reactive mixes were tested using the approach described in Methods Section 4.

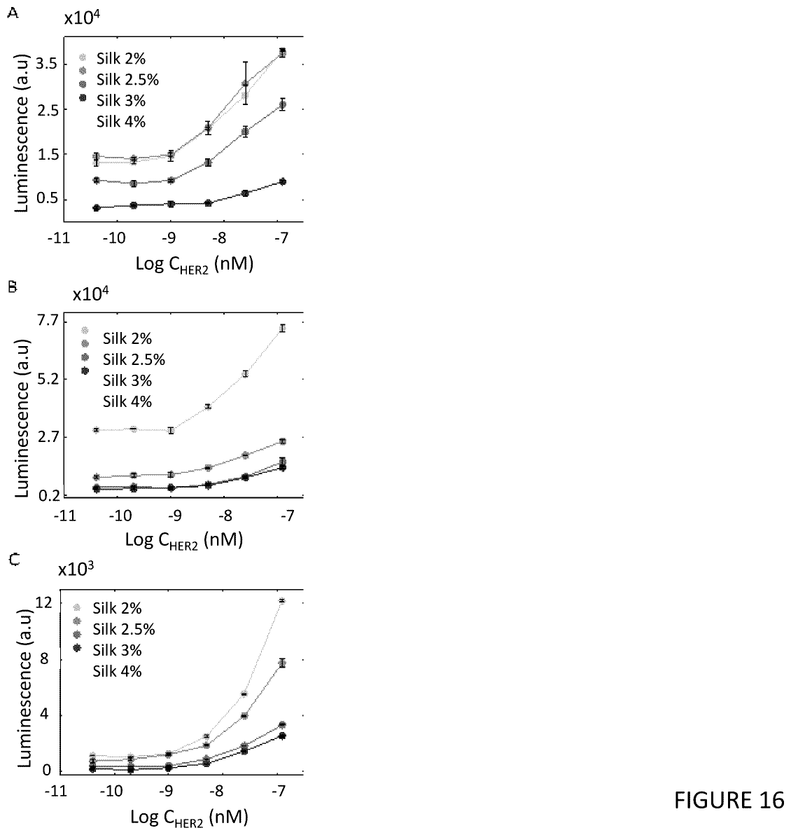

The lucKey-lucCage biosensing units maintained detecting capabilities after combination with silk fibroin solutions (FIG. 16). The chemiluminescent signal intensity was directly proportional to the concentration of the target for all RSF concentration, enabling the detection of HER2 down to nM levels (for all the different combinations, LODavg 3.5±1.6 nM).

The concentration of RSF influenced the luminescence intensity but did not compromise the overall sensing performances in terms of dynamic range and average LODs. The trend shows that the lower the amount of silk (2 wt %), the higher the luminescence intensities were. Luminescence intensities range from ˜1×10⁶ (with silk at 2 wt %) down to ˜2.6×105 counts (with silk at 4 wt %), at high target concentrations (125 nM HER2, 5 nM lucKey-lucCage, FIG. 16C). The same trend was recorded for solutions embedding lnM lucKey-lucCage (FIG. 4B). The luminescence intensities decreased overall, with a reading of 8×10⁴ counts for silk at 2 wt %, and 1.5×10⁴ counts for silk at 4 wt %. However, the lower amount of lucKey-lucCage did not affect the dynamic sensing range (5-125 nM) and LODs (LODavg=3.3±1.4 nM).

Additional testing was carried out on silk-based solutions at 4 wt %, 3 wt %, 2.5 wt %, and 2 wt % embedding 0.5 nM of lucKey-lucCage (Figure S1A). The sensing response for silk solutions at 4 wt % differs from the other mixtures during the detection of low target concentrations (range 0.04-5 nM). The luminescence intensities decreased (down to 3000-8000 counts) even with the highest target concentration (125 nM). The overall detecting performances of 3 wt %, 2.5 wt %, and 2 wt % (0.5 nM lucKey-lucCage) silk-based solutions were preserved, but luminescence counts were lower (8000-38000 counts).

It is likely that silk fibroin interacts with the lucKey-lucCage protein complex through electrostatic and hydrophobic interactions. These types of interactions are involved in the stabilizing effect of silk towards proteins and enzymes in solution, preventing protein-protein interactions and thus aggregation. In this case, higher concentrations of silk fibroin could shield the analyte binding sites of the lucKey-lucCage complex, leading to a drop in bioluminescence intensity.

### Bio-Reactive Sponges and Films

After the optimization of the lucKey-lucCage-RSF hybrid in solution, the sensing performances of the complex after incorporation in the solid-state were tested to evaluate the ability of RSF to generate shelf-stable self-standing sensing substrates (FIG. 3-10). Lyophilized sponges and films were prepared as discussed in the Methods Section 2 above. Preliminary tests were carried out to verify that the freeze-drying treatment and film casting did not compromise the functionality of the lucKey-lucCage biosensing complex. Free standing RSF formats were prepared using 2 wt % RSF and lucKey-lucCage at concentrations of (1) 5 nM for HER2 and RBD, (2) 25 nM for BoNT/B, (3) 6.25 nM for anti-HBV. Sponges and films were dissolved in water and exposed to various concentrations of analyte to evaluate potential changes in the intensity of chemiluminescent signals, using a microplate reader.

Bioluminescence response showed that the lucKey-lucCage system was stable and responsive both in the sponge (FIG. 3-6) and film formats (FIG. 7-10), as the chemiluminescent signal intensity increases with target concentrations while achieving detection within 20 minutes. The computed LODs are reported in Table 3.

TABLE 3
LODs of silk sponges and films.

| | | LOD |
|---|---|---|
| Format | Analyte | (nM) |
| Sponge | HER2 | 1 |
| | RBD | 0.005 |
| | BoNT/B | 2.4 |
| | anti-HBV | 1.5 |
| Film | HER2 | 1.2 |
| | RBD | 0.005 |
| | BoNT/B | 2.1 |
| | anti-HBV | 1.8 |

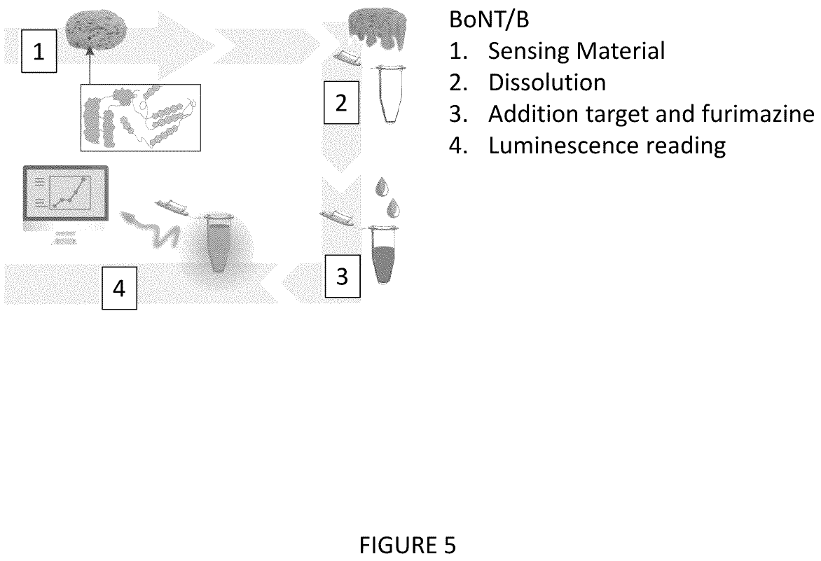

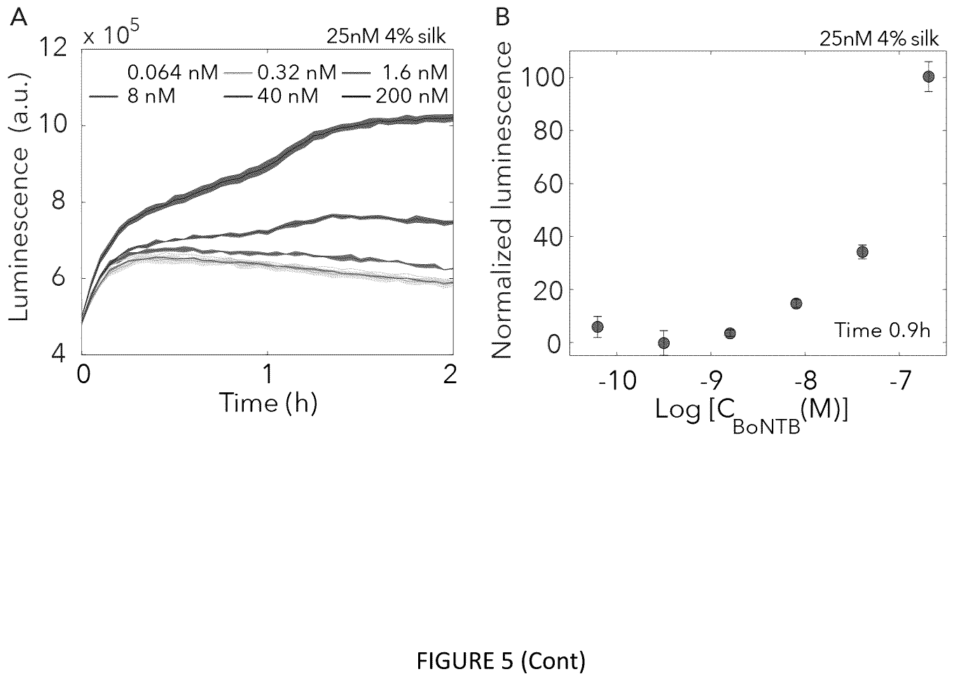

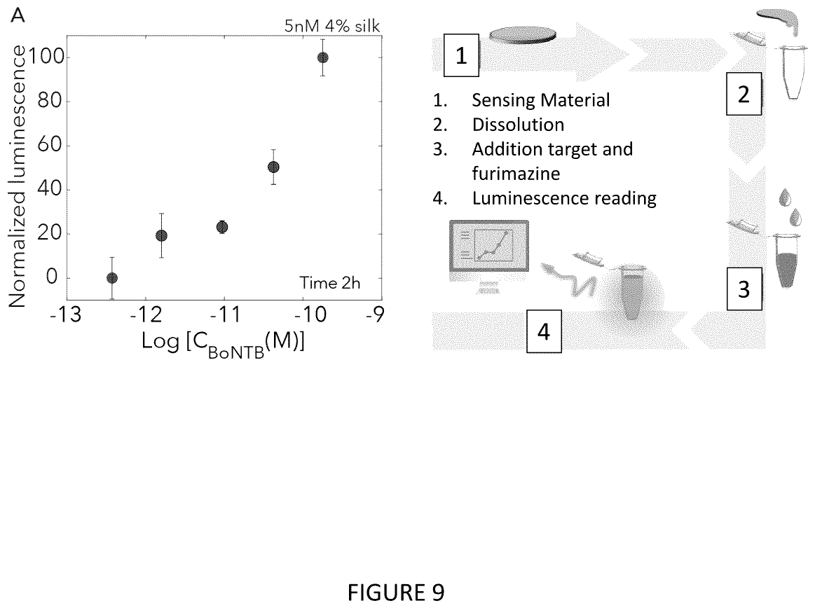

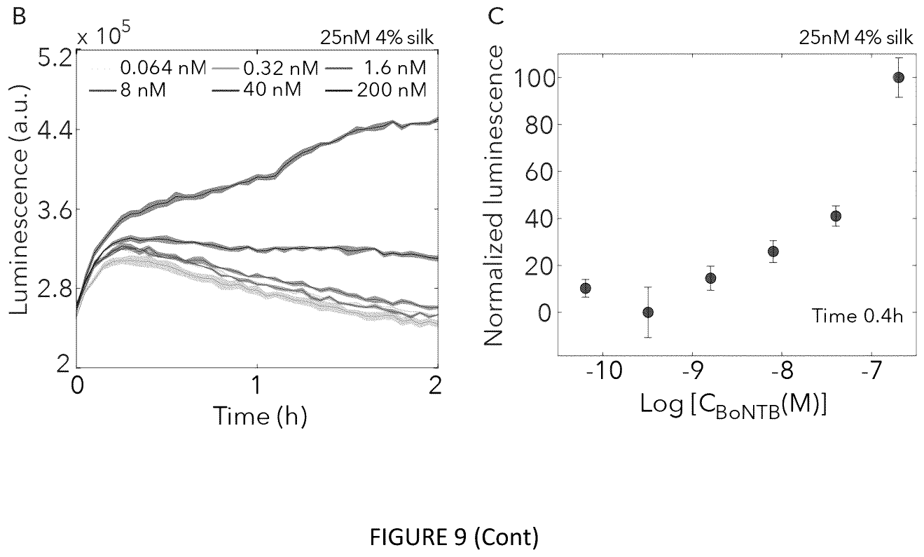

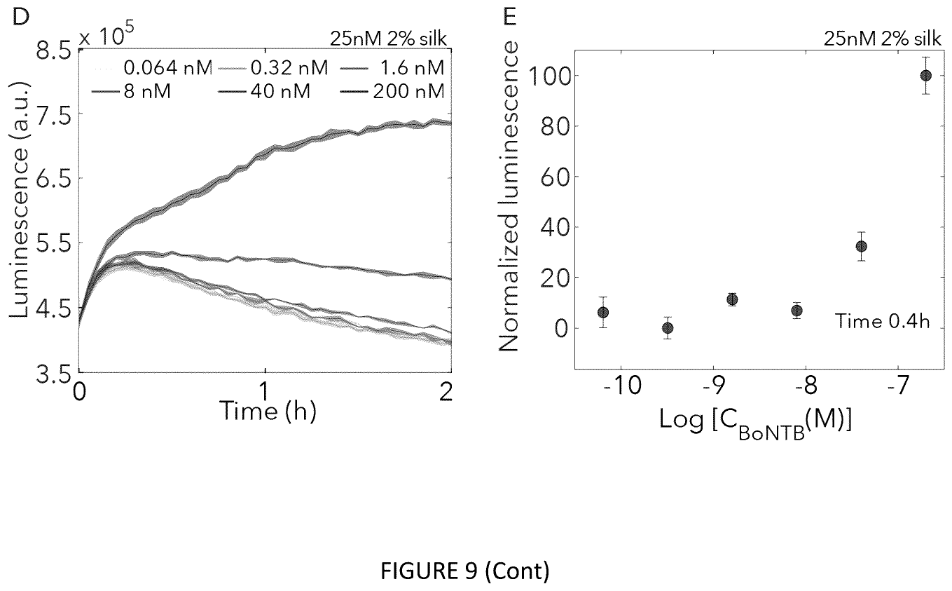

The chemiluminescent response of 4% and 2% silk sponges (FIG. 5-8) and films (FIG. 9A-D, 10F-G, 11B-C, 12B-C) showed a similar trend. For sponges, the amount of RSF directly influenced the luminescence intensity but did not compromise the sensing performance, both in terms of dynamic range and ranges of computed LODs (data reported in Table 4). Luminescence counts were still high for RSF at 4 wt %, reaching values of ˜1×10⁶ for HER2, ˜3.9×10⁵ for RBD, and ˜8×10⁵ for BoNT/B. For RSF film at 4 wt % luminescence counts reached values of ˜8.5×10⁵ for HER2, ˜3×10⁵ for RBD, for ˜4.6×10⁵ BoNT/B, and ˜1.9×10⁶ for anti-HBV.

TABLE 4
Sensing ranges and LODs for sponges
with various silk concentration.

| | | Sensing | LOD range |
|---|---|---|---|
| Format | Analyte | range (nM) | (nM) |
| Sponge | HER2 | 2.5-125 | 1-2 |
| silk 2 wt % | RBD | 0.015-0.15 | 0.005-0.01 |
| | BoNT/B | 2.5-200 | 1.5-2.4 |
| Sponge | HER2 | 2.5-125 | 0.9-2 |
| silk 4 wt % | RBD | 0.015-0.15 | 0.005-0.02 |
| | BoNT/B | 2.5-200 | 1.6-2.4 |

Different concentrations of lucKey and lucCage (1 nM and 5 nM) were tested to analyze their effect on the overall performance of HER2 and RBD detection in sponges and film formats.

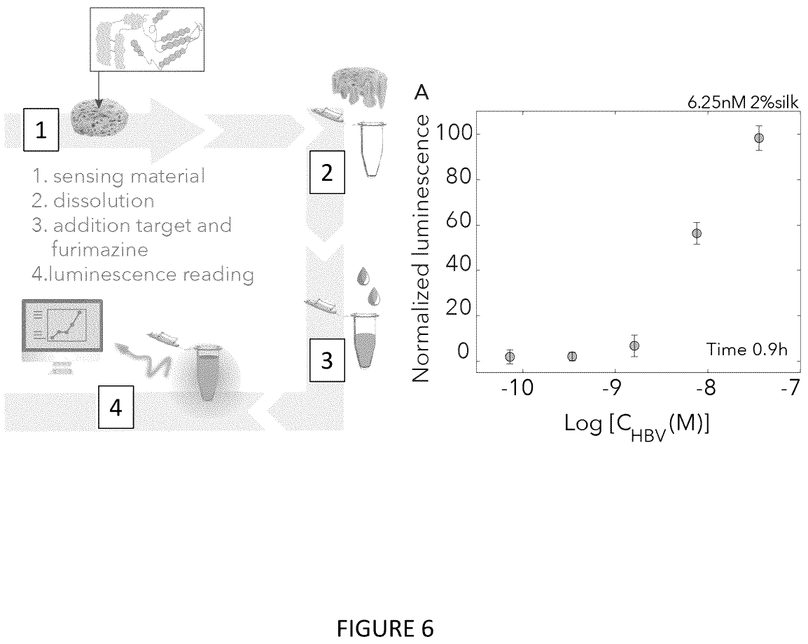

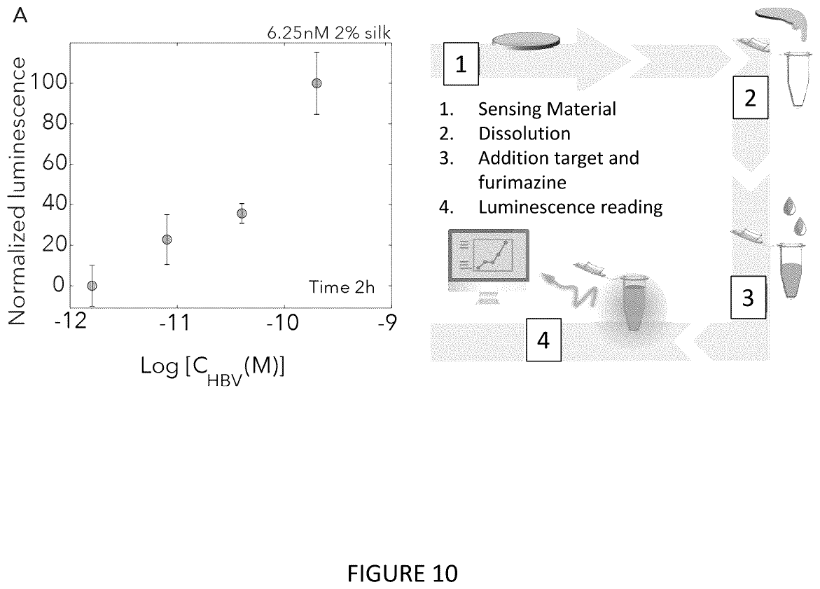

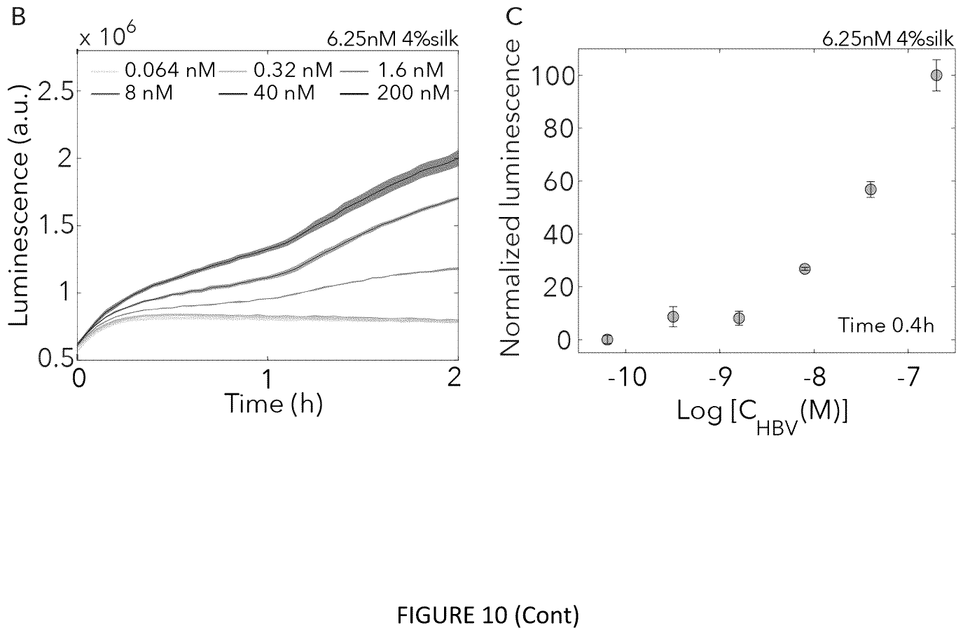

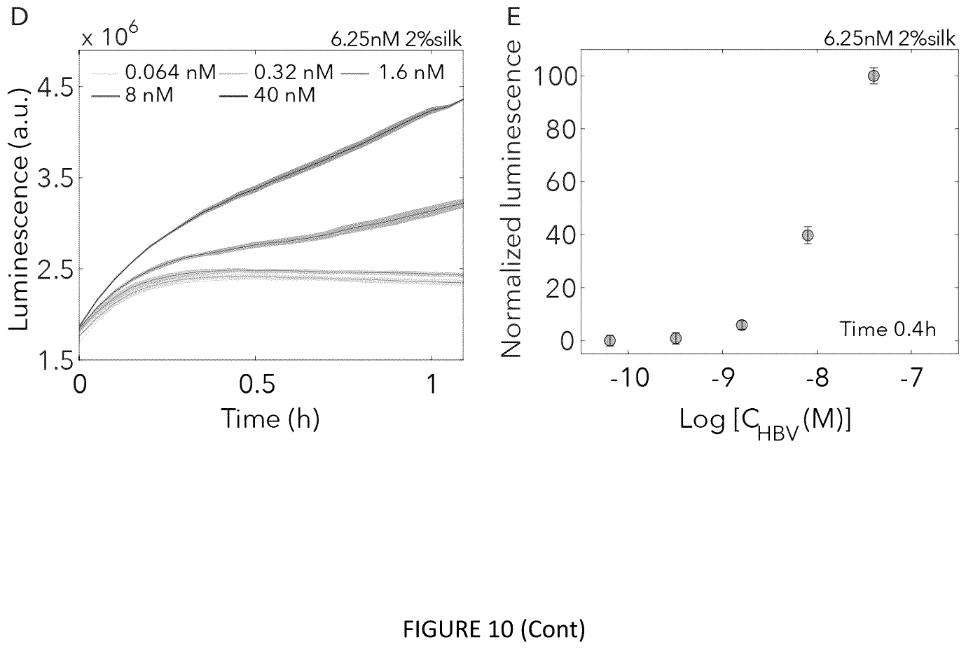

The concentration of lucKey-lucCage did not affect the dynamic sensing range (2.5-125 nM) and LOD, i.e., 1.1±0.1 for lucCageHER2 sponges (FIGS. 3B, D, F), and 1.2±0.2 for films (FIG. 7B, D, F, H). However, it influenced the performances of both the lucCageRBD sponges and films. 1 nM of lucKey-lucCageRBD complex in 4 wt % RSF solution (results not shown) did not allow reliable detection due to the high baseline noise. Although the luminescence signal was detectable for 1 nM lucKey-lucCageRBD 2 wt % RSF sponges (FIG. 6C-D) and films (FIG. 10B-C) the baseline noise was still high.

The best performances were achieved with sponges and films prepared with 5 nM lucKey-lucCageRBD and 2 wt % or 4 wt % RSF solution, which enabled accurate detection of RBD within the sensing range of 20-150 pM. For each analyte, the concentration of lucKey-lucCage was adjusted to optimize the sensing performances of the solid substrates.

The performance of the lucKey-lucCageRBD system towards the Omicron variant of the SARS-CoV-2 Spike RBD target was also tested in a 2% RSF solution. Although the system has not been optimized for the detection of Omicron, the lucKey-lucCageRBD system can detect the variant at nM concentration (FIG. 17). Further optimization of the lucKey-lucCage binding energies with the Omicron target could lead to activation of the system at the lower target concentrations.

### Bio-Reactive Films Analyzed Via Chemiluminescence Imaging

To speed up the detection method and facilitate sample analysis in non-laboratory settings, the sensing performances of responsive films were evaluated with a bioluminescence camera. Films were cast on white polystyrene substrates using the approach described in Methods Section 2 and the sensing performances of the films were assessed using a chemiluminescent imaging system (Methods Section 6, and Supplementary Methods 2). This detection method was selected since the reading unit can be potentially miniaturized into a portable device to enable delocalized monitoring outside laboratory-controlled conditions.

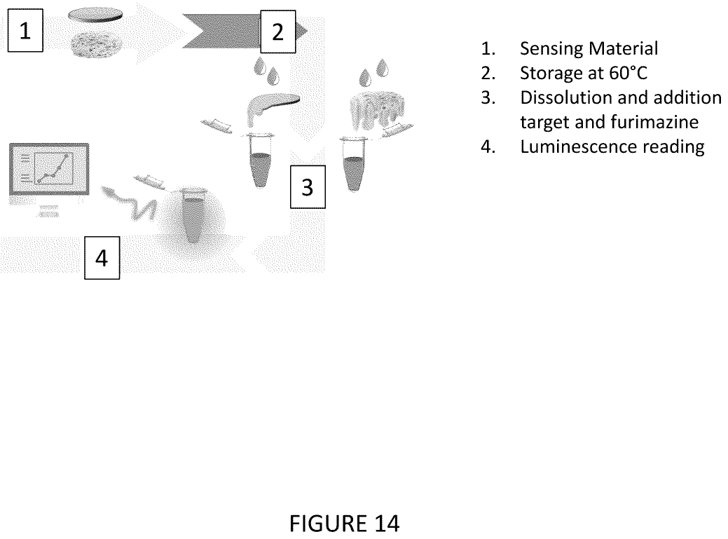

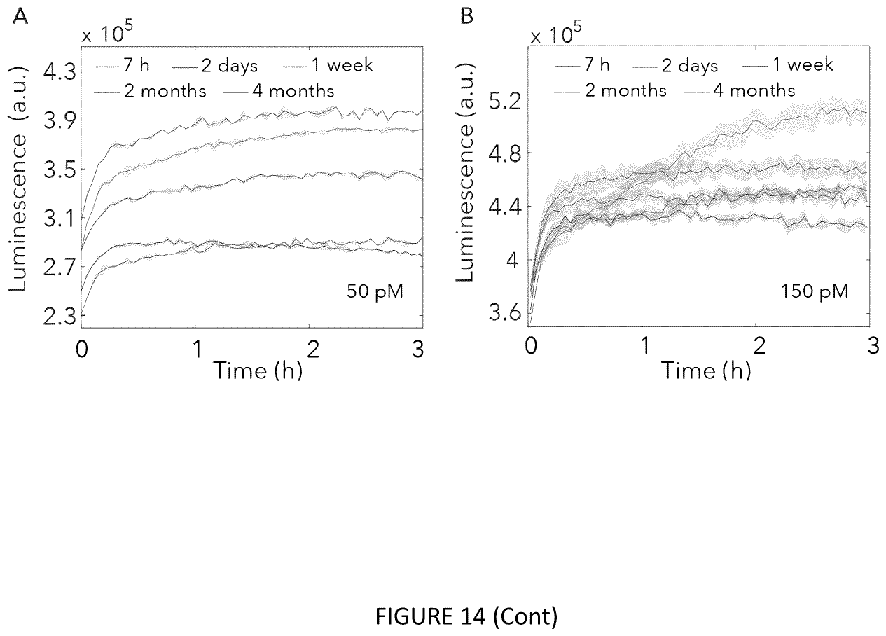

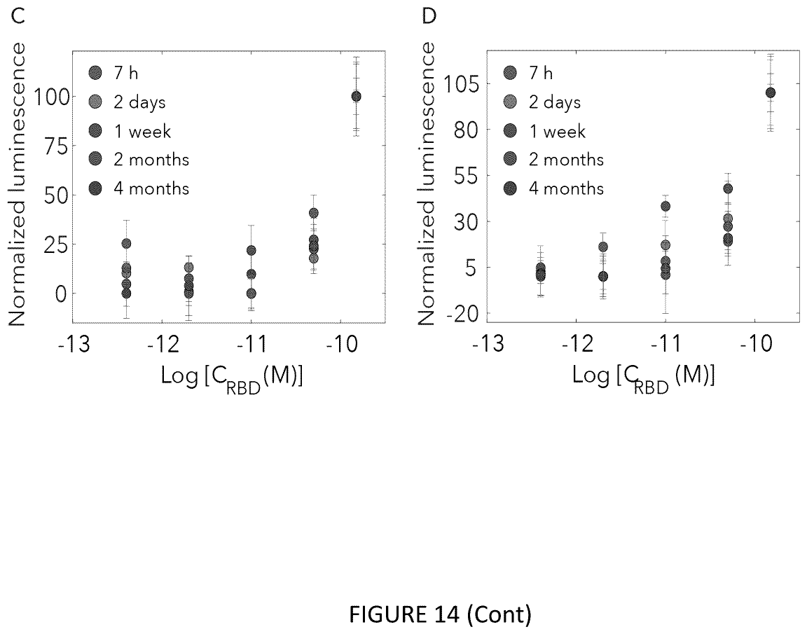

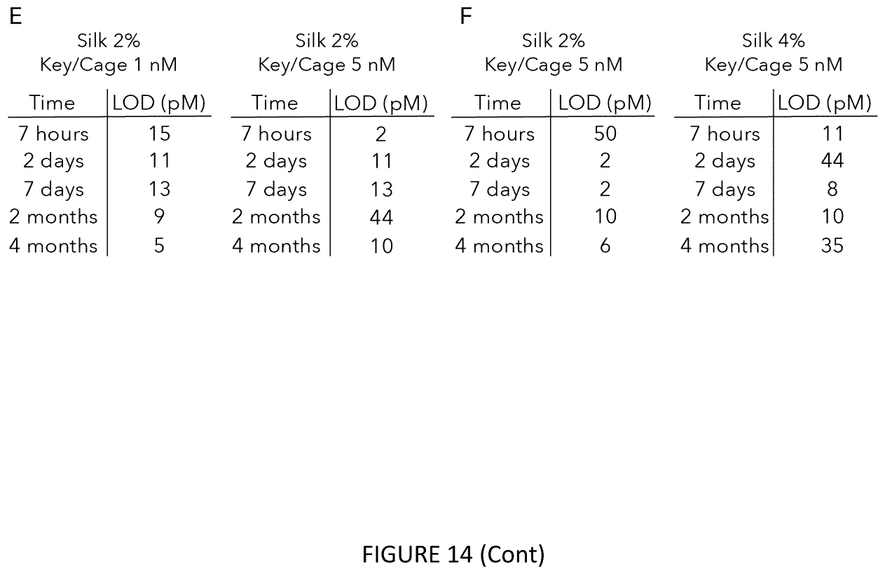

Imaging of lucKey-lucCageRBD films was achieved with the lucKey-lucCage complex at a concentration of 25 nM (FIG. 14) and allowed reaching LODs within the clinical diagnostic range expected for RBD detection (˜59 pM). The 25 nM films allowed the monitoring of RBD variations even in simulated saliva down to ˜10 pM (FIG. 2g), thus supporting the potential use of these sensing platforms as point of care devices. Responsive films were also prepared for HER2 (FIG. 2c), BoNT/B (FIG. 2e), and anti-HBV (FIG. 2i) with 25 nM, 50 nM, and 25 nM of lucKey-lucCage, which gave LOD values of ˜15 nM, ˜8.7 nM, and ˜2 nM, respectively.

To assess the stability of the system in harsh conditions, the lucKey-lucCageRBD was electrospun to obtain a non-woven mat that could have applications in air filtration units. The biosensing mix maintained its responsiveness when integrated in an electrospun mat (FIG. 12B), and the luminescence signal was still proportional to the concentration of analyte in the pM range.

### Bio-Responsive Tools Monitored Via Chemiluminescence Imaging

Bio-responsive patterns were printed or deposited onto different interfaces, including, a silicone breast pad (FIG. 3b-c, 13C), jar lid (FIG. 3d-e, 13D), a surgical mask (FIG. 3f, 13A), and nitrile gloves (FIG. 3g, 13B). Details on the procedure used for printing and imaging are provided in Supplementary Methods 5 above.

The printing process did not affect the sensing capability of the lucKey-lucCage complex, and the different printed patterns provided a chemiluminescent response proportional to various target concentrations in presence of furimazine substrate (FIG. 3, 15). Although the use of different substrates may influence the background noise of the chemiluminescence image, the signal resolution is not compromised.

The analysis of the mask (FIG. 3f, 13A) revealed a broad luminescent signal that developed around the whole printed area hampering a precise localization of the chemiluminescent response related to various RBD concentrations, likely due to the lack of control during furimazine and target spraying.

Aiming at the detection of HER2, HER2-RSF hybrid complexes were integrated onto a silicone breast pad. As most breast cancers arise from the ductal epithelium, examination of the breast ductal fluid can be used for early detection of breast cancer and to monitor and predict the efficacy of therapies. HER2 is found in nipple fluids at a higher concentration than in blood, and soluble HER-2 (whose normal concentration is in the range of ng/ml) is present in higher concentrations in nipple fluid of patients with tumors. Thus, sensing breast pads could represent a breakthrough in the early detection of breast cancer when spontaneous nipple discharge is present, as they could detect whether the discharge is benign or malign. HER2-RSF sensing pads were responsive and able to detect to different concentrations of HER2 within minutes (˜20 min) in the nM range (FIG. 3c, 13C).

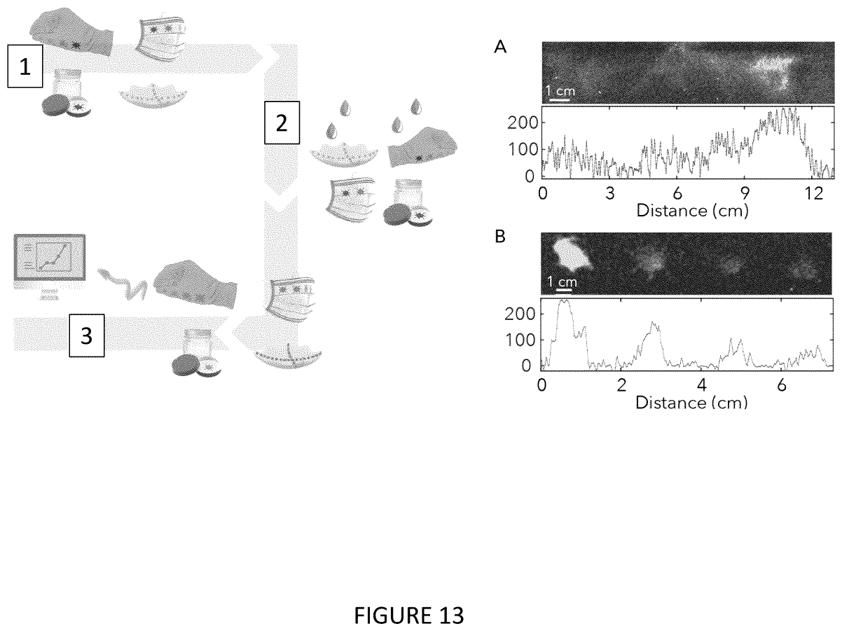

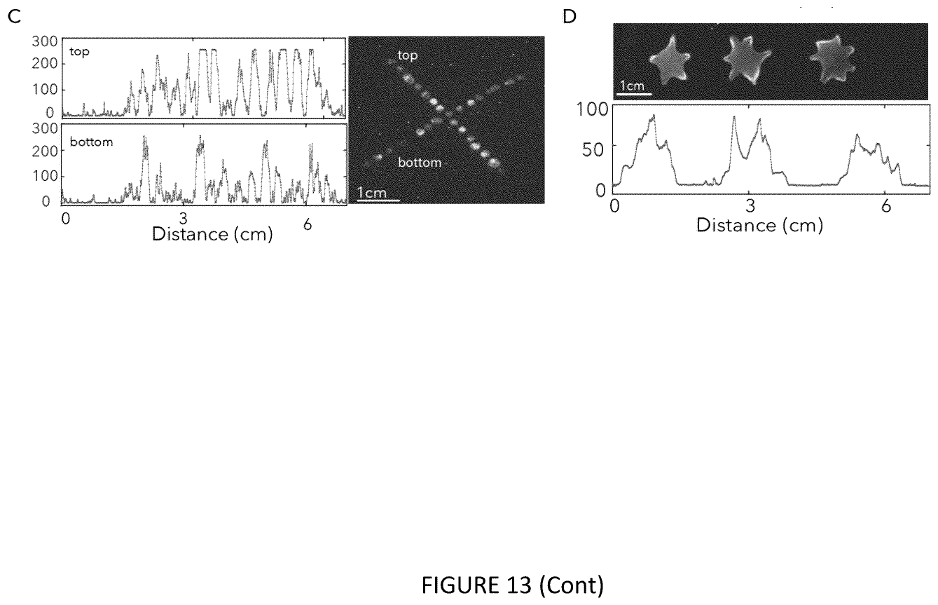

In summary, the results presented in FIGS. 3 and 13 support that the versatility of silk fibroin combined with the accuracy and specificity of the lucKey-lucCage complex enables converting common tools (such as masks and breast pads) into sensing platforms. This strategy paves the way towards responsive devices for multi-analyte detection in biofluids (e.g., salivary analytes). Moreover, these sensing devices can be adapted to other real-life scenarios, enabling the detection of air-borne viruses during sanitary crisis (e.g., SARS-CoV-2), or cancer biomarkers (e.g., HER2, marker of breast cancer) during screening or follow-up examinations. The lucKey-lucCage sensing complex can be further tuned to expand detecting approaches and enable luminescent and fluorescence readouts that may facilitate the translation into readily available miniaturized point of care devices.

### Sensing Performances of RBD Sponges and Films During Accelerated Aging Tests

Accelerated aging tests for the RBD bio-reactive films and sponges were performed to evaluate the intrinsic stability of the 2 wt % and 4 wt % silk formats embedding lucKey and lucCage at 1 and 5 nM over time (FIG. 14A). Samples were stored in an incubator at 60° C. for a total of 4 months and analyzed after 7 hours, 2 days, 7 days, 2 months, and 4 months. More precisely, films and sponges were removed from the incubator and equilibrated at room temperature. They were then completely dissolved in deionized water and analyzed with the Synergy HT Microplate Reader following the same protocol described in Methods Section 5 above.

The luminescence intensity recorded for sponges prepared with RSF at 4 wt %, and lucKey-lucCage at 5 nM decreased over time when using 50 nM RBD target concentration, with a variation of ˜25% between the first (7 hours) and final reading (4 months, FIG. 14A). The overall sensing trend, however, and the LODs were preserved over time (FIG. 14C). The same trends were recorded for RSF-based sponges (2 wt %) embedding lnM or 5 nM of lucKey-lucCage. Both sensitivity and dynamic ranges were fully preserved over time, and the LODs values (LODavg=22±4.3 pM) were stable for the different types of sponges over 4 months (FIG. 14).

Films at 2 wt % silk, 5 nM lucKey-lucCage analyzed at 150 nM of RBD concentration were found to be more stable, as luminescence intensities varied of ˜4% between the first and the last reading (FIG. 14B). The overall sensing trend and the LODs were preserved over time (FIG. 14D). Similar responses were recorded for RSF-based films (4 wt %, 2 wt %) embedding 5 nM or 1 nM of lucKey-lucCage. The sensing performances (both sensitivity and dynamic ranges) were fully preserved over time, the LODs were stable at an average value of 14±4 pM (FIG. 14F).

Some of the computed LOD values listed in FIG. 14E-F are outliers (FIG. 14E silk 2% lucKey-lucCage 5 nM, 44 pM; FIG. 14F, silk 2% lucKey-lucCage 5 nM, 50 pM; silk 4% lucKey-lucCage 5 nM, 44 pM). These unusual higher LODs can be dictated by variations in film dissolution before analysis. Nevertheless, the overall stability of the solid protein complex at 60° C. for 4 months suggests that the system is robust and preserves the detecting performances in non-refrigerated conditions, which is an important aspect for point of care platforms, yet difficult to achieve with protein-based devices.

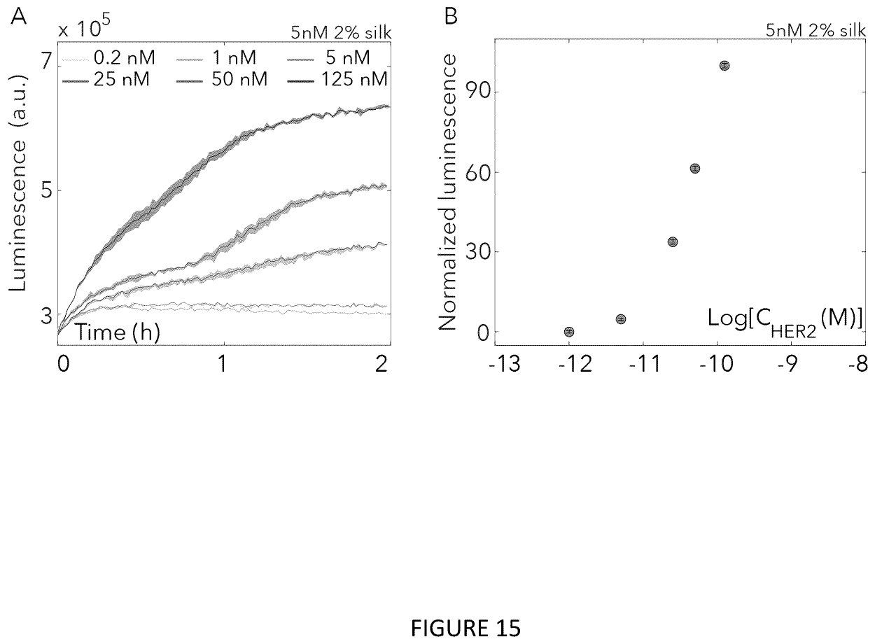

The stability of HER2-RSF hybrid sponges was re-tested after storage at room temperature (20-25° C.) over 1 year. Despite the lucKey-lucCage sensing components are typically refrigerated at −80° C., the integration in the RSF matrix preserved their sensing capabilities (FIG. 15). The HER2-RSF sponges after dissolution and exposure to target and furimazine lead to luminescence signals proportional to the concentration of analytes and enabled detection within the nM range (LOD=5 nM).

### Responsiveness of RBD Sponges to RBD-Spiked Vapor

To test the ability of the bio-responsive silk sponges to detect the SARS-CoV-2 RBD protein in air, 25 nM lucKey-lucCageRBD silk sponges were exposed to aqueous vapor with and without 500 pM of RBD target protein. The sponges were placed in a stainless-steel cylindrical chamber kept on a heating plate at 120° C. containing a water bath to generate the water vapor and exposed for 20 minutes. To prevent the direct contact of the sponges with the bath, they were placed on a metallic mesh. After the exposure to the vapor, the bioluminescence response of the sponges dissolved in deionized water was acquired for 1 h with a bench top plate reader.

Examples disclosed herein are further described in d'Amone, L., Matzeu, G., Quijano-Rubio, A., Callahan, G. P., Napier, B., Baker, D., & Omenetto, F. G. (2023). Reshaping de Novo Protein Switches into Bioresponsive Materials for Biomarker, Toxin, and Viral Detection. Advanced Materials, 35(11), 2208556 and the accompanying Supporting Information. https://doi.org/10.1002/adma.202208556, both of which are incorporated by reference in their entireties for all purposes.

1. A solid form sensing article comprising:
one or more silk fibroin matrix sensing units, wherein each of the one or more silk fibroin matrix sensing units comprises a silk fibroin matrix having entrained therein a plurality of Cage proteins and a plurality of Key proteins which form part of a Cage-Key protein system,
wherein each of the one or more silk fibroin matrix sensing units has a solid form sensing property as measured when exposing the respective silk fibroin matrix sensing unit to an analyte of interest.

2. The solid form sensing article of claim 1, wherein the plurality of Cage proteins and the plurality of Key proteins have a spatial distribution within the silk fibroin matrix that is selected from the group consisting of homogeneous distribution, random distribution, pseudo-random distribution, and combinations thereof.

3. The solid form sensing article of claim 1, wherein the silk fibroin matrix is associated with a material format, wherein the material format of the silk fibroin matrix and a concentration and distribution of the plurality of Cage proteins and the plurality of Key proteins within the silk fibroin matrix provide the solid form sensing property.

4. The solid form sensing article of claim 1, wherein the Cage-Key protein system has an intracellular sensing property as measured when exposing a cell expressing the Cage-Key protein system to the analyte of interest,
wherein the Cage-Key protein system has a solution-phase sensing property as measured when exposing a solution having dissolved therein the Cage-Key protein system to the analyte of interest.

5. The solid form sensing article of claim 1, wherein the Cage protein and the Key protein entrained in the silk fibroin matrix are not contained within a cell.

6. The solid form sensing article of claim 1, wherein the plurality of Cage proteins is present in the silk fibroin matrix in a concentration range of 1-50 nM,
wherein the plurality of Key proteins are present in the silk fibroin matrix in a concentration range of 1-50 nM,
wherein the plurality of Cage proteins and the plurality of Key proteins are present in the silk fibroin matrix in a ratio of: 0.13-1.3% w/w for the plurality of Cage proteins; 0.082-0.82% w/w for the plurality of Key proteins; 22.5-80% w/w for the silk fibroin considering an initial weight of a matrix solution of Key proteins, Cage proteins, and silk fibroin, wherein the matrix solution provides the silk fibroin matrix by loss of water.

7. (canceled)

8. The solid form sensing article of claim 1, wherein the silk fibroin matrix material format is selected from the group consisting of sponge, film, foam, printable ink, and electrospun mat, and combinations thereof.

9. The solid form sensing article of claim 1, wherein the Cage protein is lucCage, the Key protein is lucKey, and the Cage-Key protein system is the lucCage-lucKey protein system.

10. The solid form sensing article of claim 1, wherein the analyte of interest is the receptor-binding domain (RBD) in Severe Acute Respiratory Syndrome Coronavirus 2 (SARS-CoV-2) spike protein, the Human Epidermal Growth Factor Receptor 2 (HER2), the Botulinum Neurotoxin B (BoNT/B), or the Anti-Hepatitis B Virus antibody (anti-HBV).

11.-13. (canceled)

14. The solid form sensing article of claim 1, wherein the article is a breast pad.

15. The solid form sensing article of claim 14, wherein at least one of the one or more silk fibroin matrix sensing units is positioned at a nipple-contacting portion, an areola-contacting portion, a skin-contacting portion of the breast pad, wherein the nipple-contacting portion contacts a nipple of a user when the breast pad is in use, wherein the areola-receiving portion contacts an areola of a user when the breast pad is in use, wherein the skin-contacting portion contacts a portion of non-nipple, non-areola skin located on a breast of a user when the breast pad is in use.

16. The solid form sensing article of claim 1, wherein the article is an aerial drone.

17. The solid form sensing article of claim 1, wherein the article is a glove, a mask, a sticker, an article of clothing, and/or a container.

18.-23. (canceled)

24. The solid form sensing article of claim 1, wherein the one or more silk fibroin matrix sensing units is two or more silk fibroin matrix sensing units, wherein at least two of the two or more silk fibroin matrix sensing units have different Cage-Key protein systems tuned to different analytes of interest.

25. (canceled)

26. The solid form sensing article of claim 1, wherein the two or more silk fibroin sensing units is twenty-five or more silk fibroin sensing units, wherein at least twenty-five of the twenty-five or more silk fibroin sensing units have different Cage-Key protein systems tuned to different analytes of interest.

27. The solid form sensing article of claim 1, wherein the solid form sensing article provides a picomolar limit of detection for the analyte of interest.

28. The solid form sensing article of claim 1, wherein the solid form sensing article maintains at least 90% of sensing performance after storage at a temperature of 25° C. at relative humidity in the range of 25-40% for 4 months.

29. A method of using the solid form sensing article of claim 1, the method comprising: monitoring a Cage-Key signal associated with the presence of the analyte of interest.

30. A method of making a solid form sensing article, the method comprising: drop casting liquid silk fibroin-key/cage matrices converted into sensitive films after water evaporation.

31. A method of making a solid form sensing article, the method comprising: lyophilizing silk fibroin-key/cage matrices to make sponges.

32. (canceled)

33. (canceled)
